(19) United States
(12) Patent Application Publication
(10) Pub. No.: US 2008/0292576 A1
(43) Pub. Date: Nov. 27, 2008

(54) METHOD FOR IDENTIFYING HAIR CONDITIONER-RESISTANT HAIR-BINDING PEPTIDES AND HAIR BENEFIT AGENTS THEREFROM

(75) Inventors: HONG WANG, Kennett Square, PA (US); Ying Wu, Wallingford, PA (US); John P. O'Brien, Oxford, PA (US)

Correspondence Address: E I DU PONT DE NEMOURS AND COMPANY;LEGAL PATENT RECORDS CENTER, BARLEY MILL PLAZA 25/1122B, 4417 LANCASTER PIKE, WILMINGTON, DE, 19805, US

(73) Assignee: E.I. du Pont de Nemours and Co., Wilmington, DE (US)

(21) Appl. No.: 12/144,907

(22) Filed: Jun. 24, 2008

Related U.S. Application Data
(62) Division of application No. 11/359,163, filed on Feb. 22, 2006, now abandoned.
(60) Provisional application No. 60/657,496, filed on Mar. 1, 2005.

(51) Int. Cl. A61K 8/64; C40B 30/04; C07K 2/00; A61Q 5/12; C07K 14/00; C07K 7/00

(52) U.S. Cl. 424/70.14; 506/9; 530/300; 530/324; 530/326; 8/405

(57) ABSTRACT

A method for identifying hair conditioner-resistant hair-binding peptides is described. The hair conditioner-resistant hair-binding peptides bind strongly to hair from a hair conditioner matrix and are stable therein. Peptide-based benefit agents, such as hair conditioners and hair colorants, based on the hair conditioner-resistant hair binding peptides are described. The peptide-based hair conditioners and hair colorants consist of a hair conditioner-resistant hair-binding peptide coupled to a hair conditioning agent or a coloring agent, either directly or through an optional spacer. Hair care and hair coloring product compositions comprising these peptide-based hair conditioners and colorants are also described.

This patent application claims the benefit of United States Provisional Patent Application, 60/657,496, filed Mar. 1, 2005.

### FIELD OF THE INVENTION

The invention relates to the field of personal care products. More specifically, the invention relates to a method for identifying hair conditioner-resistant hair-binding peptides and the use thereof in peptide-based hair benefit agents, such as hair conditioners and colorants.

### BACKGROUND OF THE INVENTION

Hair conditioners and hair colorants are well-known and frequently used hair care products. The major problem with current hair conditioners and non-oxidative hair dyes is that they lack the required durability for long-lasting effects. Oxidative hair dyes provide long-lasting color, but the oxidizing agents they contain cause hair damage. In order to improve the durability of these compositions, peptide-based hair conditioners, hair colorants, and other benefit agents have been developed (Huang et al., copending and commonly owned U.S. Patent Application Publication No. 2005/0050656, and U.S. Patent Application Publication No. 2005/0226839). The peptide-based hair conditioners or colorants are prepared by coupling a specific peptide sequence that has a high binding affinity to hair with a conditioning or coloring agent, respectively. The peptide portion binds to the hair, thereby strongly attaching the conditioning or coloring agent. Peptides with a high binding affinity to hair have been identified using phage display screening techniques (Huang et al., supra; Estell et al. WO 0179479; Murray et al., U.S. Patent Application Publication No. 2002/0098524; Janssen et al., U.S. Patent Application Publication No. 2003/0152976; and Janssen et al., WO 04048399). The 0179479, 2002/0098524, 2003/0152976, and 04048399 applications describe contacting a peptide library with a hair sample in the presence of a dilute solution of bath gel (i.e., a 2% aqueous solution) and washing the phage-peptide-hair complex with the bath gel solution during phage display screening; however, the concentration of bath gel used is too low to identify bath gel-resistant hair-binding peptides.

The hair-binding peptides have decreased binding affinity in the presence of a hair conditioner matrix and therefore do not bind strongly to hair from the conditioner matrix or are washed from the hair by the application of a hair conditioner. Moreover, the hair-binding peptides are not stable for long periods of time in the conditioner matrix, which causes their binding affinity to decrease with time in the hair conditioner product.

Methods for identifying shampoo-resistant hair-binding peptides (Huang et al., copending and commonly owned U.S. Patent Application Publication No. 2005/0050656, and O'Brien et al., copending and commonly owned U.S. patent application Ser. No. 11/251,715), shampoo-resistant antibody fragments that bind to a cell surface protein of Malassezia furfur (Dolk et al., Appl. Environ. Microbiol. 71:442-450 (2005)), and skin care composition-resistant skin binding peptides (Wang et al., copending and commonly owned U.S. Patent Application No. 60/657,494) have been reported. However, methods for identifying hair conditioner-resistant hair-binding peptides have not been described.

The problem to be solved, therefore, is to provide hair-binding peptides that are able to bind to hair from a hair conditioner matrix and are stable therein.

Applicants have addressed the stated problem by discovering a method for identifying hair conditioner-resistant hair-binding peptides. The identified hair conditioner-resistant hair-binding peptide sequences bind to hair from a hair conditioner matrix and show no loss in binding activity after a period of 21 days in the conditioner matrix. These hair-binding peptides may be used to prepare peptide-based hair benefit agents, such as hair conditioners and colorants, having high binding affinity to hair in the presence of a hair conditioner matrix and improved stability in a hair conditioner composition.

### SUMMARY OF THE INVENTION

The invention provides methods for the identification and isolation of new hair-conditioner resistant hair-binding peptides useful as linkers and adhesives in hair care compositions. The hair-conditioner resistant hair-binding peptides may be incorporated in diblock or triblock structures optionally comprising chemical or peptide spacers and benefit agents, such as colorants and/or conditioners. The methods of the invention rely on the screening of combinatorially generated peptide libraries for hair binding properties in the presence of various conditioning agents.

Accordingly the invention provides a method for identifying a hair conditioner-resistant hair-binding peptide comprising:
a) providing a combinatorial library of DNA associated peptides;
b) contacting the library of (a) with a hair sample to form a reaction solution comprising DNA associated peptide-hair complexes;
c) isolating the DNA associated peptide-hair complexes of (b) from the reaction solution;
d) contacting the isolated DNA associated peptide-hair complexes of (c) with a hair conditioner matrix to form a conditioning solution wherein the concentration of the hair conditioner matrix is at least about 10% of full strength concentration;
e) isolating the DNA associated peptide-hair complexes of (d) from the conditioning solution;
f) amplifying the DNA encoding the peptide portion of the DNA associated peptide-hair complexes of (e); and
g) sequencing the amplified DNA of (f) encoding a conditioner resistant hair-binding peptide wherein the conditioner-resistant hair-binding peptide is identified.

Optionally the hair-binding peptides may be eluted from the hair with an eluting agent after step (e) and peptides identified by the method of the invention may be further refined by successive applications to the method.

Additionally the invention provides a hair conditioner-resistant hair-binding peptide identified by a process comprising the steps of:
a) providing a combinatorial library of DNA associated peptides;
b) contacting the library of (a) with a hair sample to form a reaction solution comprising DNA associated peptide-hair complexes;
c) isolating the DNA associated peptide-hair complexes of (b) from the reaction solution;
d) contacting the isolated DNA associated peptide-hair complexes of (c) with a hair conditioner matrix to form a conditioning solution wherein the concentration of the hair conditioner matrix is at least about 10% of full strength concentration;
e) isolating the DNA associated peptide-hair complexes of (d) from the conditioning solution;
f) amplifying the DNA encoding the peptide portion of the DNA associated peptide-hair complexes of (e); and
g) sequencing the amplified DNA of (f) encoding a conditioner resistant hair-binding peptide wherein the conditioner-resistant hair-binding peptide is identified. Specific hair conditioner-resistant hair-binding peptides of the invention are set forth in SEQ ID NO:1, SEQ ID NO:2, SEQ ID NO:3, SEQ ID NO:4, SEQ ID NO:5, and SEQ ID NO:12.

In one embodiment the invention provides a diblock, peptide-based benefit agent having the general structure (HCPₘ)ₙ—BA, wherein
a) HCP is a hair conditioner-resistant hair-binding peptide;
b) BA is a benefit agent;
c) m ranges from 1 to about 100; and
d) n ranges from 1 to about 50,000.

In similar fashion, the invention provides a triblock, peptide-based benefit agent having the general structure [(HCP_{X}—S)ₘ]ₙ—BA, wherein
a) HCP is a hair conditioner-resistant hair-binding peptide;
b) BA is a benefit agent;
c) S is a spacer;
d) x ranges from 1 to about 10;
e) m ranges from 1 to about 100; and
f) n ranges from 1 to about 50,000.

In a preferred embodiment the invention provides hair conditioners and colorants where the hair conditioner-resistant hair-binding peptide is isolated by a process comprising the steps of:
a) providing a combinatorial library of DNA associated peptides;
b) contacting the library of (a) with a hair sample to form a reaction solution comprising DNA associated peptide-hair complexes;
c) isolating the DNA associated peptide-hair complexes of (b) from the reaction solution;
d) contacting the isolated DNA associated peptide-hair complexes of (c) with a hair conditioner matrix to form a conditioning solution wherein the concentration of the hair conditioner matrix is at least about 10% of full strength concentration;
e) isolating the DNA associated peptide-hair complex of (d) from the conditioning solution;
f) amplifying the DNA encoding the peptide portion of the DNA associated peptide-hair complex of (e); and
g) sequencing the amplified DNA of (f) encoding a conditioner resistant hair-binding peptide wherein the conditioner-resistant hair-binding peptide is identified.

Additionally the invention provides hair care product compositions comprising effective amounts of the peptide diblock and triblock benefit agents of the invention.

In another embodiment the invention provides a method for forming a protective layer of a peptide-based conditioner on hair comprising applying the composition of the invention to the hair and allowing the formation of said protective layer. Similarly the invention provides a method for coloring hair comprising applying the composition of the invention to the hair for a period of time sufficient to cause coloration of the hair. Alternatively, the invention provides a method for coloring eyebrows or eyelashes comprising applying the composition of the invention to eyebrow or eyelashes.

In another embodiment the invention provides a method for coloring hair, eyebrows or eyelashes comprising the steps of:
a) providing a hair coloring composition comprising a hair colorant selected from the group consisting of:
   i) (HCPₘ)ₙ—C; and
   ii) [(HCPₓ—S)ₘ]ₙ—C
      wherein:
      1) HCP is a hair conditioner-resistant hair-binding peptide;
      2) C is a coloring agent;
      3) n ranges from 1 to about 50,000;
      4) S is a spacer;
      5) m ranges from 1 to about 100; and
      6) x ranges from 1 to about 10;
      and wherein the hair conditioner-resistant hair-binding peptide is selected by a method comprising the steps of:
         A) providing a combinatorial library of DNA associated peptides;
         B) contacting the library of (A) with a hair sample to form a reaction solution comprising DNA associated peptide-hair complexes;
         C) isolating the DNA associated peptide-hair complexes of (B) from the reaction solution;
         D) contacting the isolated DNA associated peptide-hair complexes of (C) with a hair conditioner matrix to form a conditioning solution wherein the concentration of the hair conditioner matrix is at least about 10% of full strength concentration;
         E) isolating the DNA associated peptide-hair complexes of (D) from the conditioning solution;
         F) amplifying the DNA encoding the peptide portion of the DNA associated peptide-hair complexes of (E); and
         G) sequencing the amplified DNA of (F) encoding a hair conditioner resistant hair-binding peptide wherein the hair conditioner-resistant hair-binding peptide is identified; and
b) applying the hair coloring composition of (a) to hair, eyebrows or eyelashes for a time sufficient for the hair colorant to bind to hair, eyebrows or eyelashes.

Similarly the invention provides a method for forming a protective layer of a peptide-based conditioner on hair comprising the steps of:
a) providing a hair care composition comprising a hair conditioner selected from the group consisting of:
   i) (HCPₘ)ₙ—HCA; and
   ii) [(HCP_{X}—S)ₘ]ₙ—HCA
      wherein:
      1) HCP is a hair conditioner-resistant hair-binding peptide;
      2) HCA is a hair conditioning agent;
      3) n ranges from 1 to about 50,000;
      4) S is a spacer;
      5) m ranges from 1 to about 100; and
      6) x ranges from 1 to about 10;
      and wherein the hair conditioner-resistant hair-binding peptide is selected by a method comprising the steps of:
         A) providing a combinatorial library of DNA associated peptides;
         B) contacting the library of (A) with a hair sample to form a reaction solution comprising DNA associated peptide-hair complexes;
         C) isolating the DNA associated peptide-hair complexes of (B) from the reaction solution;
         D) contacting the isolated DNA associated peptide-hair complexes of (C) with a hair conditioner matrix to form a conditioning solution wherein the concentration of the hair conditioner matrix is at least about 10% of full strength concentration;
         E) isolating the DNA associated peptide-hair complexes of (D) from the conditioning solution;
         F) amplifying the DNA encoding the peptide portion of the DNA associated peptide-hair complexes of (E); and
         G) sequencing the amplified DNA of (F) encoding a conditioner resistant hair-binding peptide wherein the conditioner-resistant hair-binding peptide is identified; and
b) applying the hair care composition of (a) to hair and allowing the formation of said protective layer.

### BRIEF DESCRIPTION OF THE FIGURES AND SEQUENCE DESCRIPTIONS

The invention can be more fully understood from the following detailed description, figures and the accompanying sequence descriptions, which form a part of this application.

FIG. 1 shows the stability of the hair conditioner-resistant hair-binding peptide HCP.1 (SEQ ID NO:1) in a hair conditioner matrix.

FIG. 2 shows the stability of the hair conditioner-resistant hair-binding peptide HCP.6 (SEQ ID NO:4) in a hair conditioner matrix.

The following sequences conform with 37 C.F.R. 1.821-1.825 (“Requirements for Patent Applications Containing Nucleotide Sequences and/or Amino Acid Sequence Disclosures—the Sequence Rules”) and consistent with World Intellectual Property Organization (WIPO) Standard ST.25 (1998) and the sequence listing requirements of the EPO and PCT (Rules 5.2 and 49.5(a-bis), and Section 208 and Annex C of the Administrative Instructions). The symbols and format used for nucleotide and amino acid sequence data comply with the rules set forth in 37 C.F.R. §1.822.

SEQ ID NOs:1-5 are the amino acid sequences of hair conditioner-resistant hair-binding peptides.

SEQ ID NO:6 is the amino acid sequence of the Caspase 3 cleavage site.

SEQ ID NO:7 is the nucleotide sequence of the oligonucleotide primer used to sequence phage DNA.

SEQ ID NO:8 is the amino acid sequence of a skin-binding peptide used as a control in Example 4.

SEQ ID NO:9 is the amino acid sequence of hair conditioner-resistant hair binding peptide HCP.1(5-FAM), which has been derivatized with the fluorescent tag 5-carboxyfluorescein-aminohexyl amidite at the C-terminus, as described in Example 4.

SEQ ID NO:10 is the amino acid sequence of hair conditioner-resistant hair binding peptide HCP.6(5-FAM), which has been derivatized with the fluorescent tag 5-carboxyfluorescein-aminohexyl amidite at the C-terminus, as described in Example 4.

SEQ ID NO:11 is the amino acid sequence of the skin-binding control peptide Skin 1(5-FAM), which has been derivatized with the fluorescent 5-carboxyfluorescein-aminohexyl amidite at the C-terminus, as described in Example 4.

SEQ ID NO:12 is the amino acid sequence of the cysteine-attached HCP.1 hair-binding peptide described in Example 8.

SEQ ID NOs:13-15 are the amino acid sequences of peptide spacers.

### DETAILED DESCRIPTION OF THE INVENTION

The invention provides a method for identifying hair conditioner-resistant peptide sequences that specifically bind to human hair with high affinity in the presence of a hair conditioner matrix. The identified hair conditioner-resistant hair-binding peptide sequences bind to hair from a hair conditioner matrix and show no loss in binding activity after a period of 21 days in the conditioner matrix. These hair-binding peptides may be used to prepare peptide-based hair benefit agents, such as hair conditioners and colorants, having high binding affinity to hair in the presence of a hair conditioner matrix and improved stability in a hair conditioner composition.

The following definitions are used herein and should be referred to for interpretation of the claims and the specification.

“HCP” means hair conditioner-resistant hair-binding peptide.

“BA” means hair benefit agent.

“HCA” means hair conditioning agent.

“C” means hair coloring agent.

“S” means spacer.

The term “peptide” refers to two or more amino acids joined to each other by peptide bonds or modified peptide bonds.

The term “hair” as used herein refers to human hair, eyebrows, and eyelashes.

The phrase “hair conditioner-resistant hair-binding peptide” refers to a peptide that binds strongly to hair from a hair conditioner matrix and is stable therein.

The phrase “hair conditioner matrix” refers to a medium comprising a hair conditioner product, either undiluted or in diluted form, or a mixture comprising at least one component of a hair conditioner product, in addition, at least two components of a hair conditioner product. Components of hair conditioner products include, but are not limited to, hair conditioning agents, antioxidants, preserving agents, fillers, surfactants, UVA and/or UVB sunscreens, fragrances, thickeners, wetting agents, and anionic, nonionic or amphoteric polymers; and dyes or pigments.

The phrase “full strength concentration” refers to the concentration of components as they occur in a hair conditioner product.

The term “benefit agent” is a general term referring to a compound or substance that may be coupled with a hair conditioner-resistant hair-binding peptide for application to hair to provide a cosmetic or dermatological effect. Benefit agents typically include conditioners, colorants, fragrances, sunscreens, and the like along with other substances commonly used in the personal care industry.

The terms “coupling” and “coupled” as used herein refer to any chemical association and includes both covalent and non-covalent interactions.

The term “peptide-hair complex” means structure comprising a peptide bound to a hair fiber via a binding site on the peptide.

The term “DNA associated peptide-hair complex” refers to a complex between hair and a peptide where the peptide has associated with it an identifying nucleic acid component. Typically, the DNA associated peptide is produced as a result of a display system such as phage display. In this system, peptides are displayed on the surface of the phage while the DNA encoding the peptides is contained within the attached glycoprotein coat of the phage. The association of the coding DNA within the phage may be used to facilitate the amplification of the coding region for the identification of the peptide.

The term “non-target” refers to a substrate for which peptides with a binding affinity thereto are not desired. For the selection of hair conditioner-resistant hair-binding peptides, non-targets, include, but are not limited to, skin and plastic.

The term “nanoparticles” is herein defined as particles with an average particle diameter of between 1 and 100 nm. Preferably, the average particle diameter of the particles is between about 1 and 40 nm. As used herein, “particle size” and “particle diameter” have the same meaning. Nanoparticles include, but are not limited to, metallic, semiconductor, polymer, or other organic or inorganic particles.

The term “amino acid” refers to the basic chemical structural unit of a protein or polypeptide. The following abbreviations are used herein to identify specific amino acids:

| | Three-Letter | One-Letter |
|---|---|---|
| Amino Acid | Abbreviation | Abbreviation |
| Alanine | Ala | A |
| Arginine | Arg | R |
| Asparagine | Asn | N |
| Aspartic acid | Asp | D |
| Cysteine | Cys | C |
| Glutamine | Gln | Q |
| Glutamic acid | Glu | E |
| Glycine | Gly | G |
| Histidine | His | H |
| Isoleucine | Ile | I |
| Leucine | Leu | L |
| Lysine | Lys | K |
| Methionine | Met | M |
| Phenylalanine | Phe | F |
| Proline | Pro | P |
| Serine | Ser | S |
| Threonine | Thr | T |
| Tryptophan | Trp | W |
| Tyrosine | Tyr | Y |
| Valine | Val | V |

“Gene” refers to a nucleic acid fragment that expresses a specific protein, including regulatory sequences preceding (5′ non-coding sequences) and following (3′ non-coding sequences) the coding sequence. “Native gene” refers to a gene as found in nature with its own regulatory sequences “Chimeric gene” refers to any gene that is not a native gene, comprising regulatory and coding sequences that are not found together in nature. Accordingly, a chimeric gene may comprise regulatory sequences and coding sequences that are derived from different sources, or regulatory sequences and coding sequences derived from the same source, but arranged in a manner different than that found in nature. A “foreign” gene refers to a gene not normally found in the host organism, but that is introduced into the host organism by gene transfer. Foreign genes can comprise native genes inserted into a non-native organism, or chimeric genes.

“Synthetic genes” can be assembled from oligonucleotide building blocks that are chemically synthesized using procedures known to those skilled in the art. These building blocks are ligated and annealed to form gene segments which are then enzymatically assembled to construct the entire gene. “Chemically synthesized”, as related to a sequence of DNA, means that the component nucleotides were assembled in vitro. Manual chemical synthesis of DNA may be accomplished using well-established procedures, or automated chemical synthesis can be performed using one of a number of commercially available machines. Accordingly, the genes can be tailored for optimal gene expression based on optimization of nucleotide sequence to reflect the codon bias of the host cell. The skilled artisan appreciates the likelihood of successful gene expression if codon usage is biased towards those codons favored by the host. Determination of preferred codons can be based on a survey of genes derived from the host cell where sequence information is available.

“Coding sequence” refers to a DNA sequence that codes for a specific amino acid sequence. “Suitable regulatory sequences” refer to nucleotide sequences located upstream (5′ non-coding sequences), within, or downstream (3′ non-coding sequences) of a coding sequence, and which influence the transcription, RNA processing or stability, or translation of the associated coding sequence. Regulatory sequences may include promoters, translation leader sequences, introns, polyadenylation recognition sequences, RNA processing site, effector binding site and stem-loop structure.

“Promoter” refers to a DNA sequence capable of controlling the expression of a coding sequence or functional RNA. In general, a coding sequence is located 3′ to a promoter sequence. Promoters may be derived in their entirety from a native gene, or be composed of different elements derived from different promoters found in nature, or even comprise synthetic DNA segments. It is understood by those skilled in the art that different promoters may direct the expression of a gene in different tissues or cell types, or at different stages of development, or in response to different environmental or physiological conditions. Promoters which cause a gene to be expressed in most cell types at most times are commonly referred to as “constitutive promoters”. It is further recognized that since in most cases the exact boundaries of regulatory sequences have not been completely defined, DNA fragments of different lengths may have identical promoter activity.

The term “expression”, as used herein, refers to the transcription and stable accumulation of sense (mRNA) or antisense RNA derived from the nucleic acid fragment of the invention. Expression may also refer to translation of mRNA into a polypeptide.

The term “transformation” refers to the transfer of a nucleic acid fragment into the genome of a host organism, resulting in genetically stable inheritance. Host organisms containing the transformed nucleic acid fragments are referred to as “transgenic” or “recombinant” or “transformed” organisms.

The term “host cell” refers to cell which has been transformed or transfected, or is capable of transformation or transfection by an exogenous polynucleotide sequence.

The terms “plasmid”, “vector” and “cassette” refer to an extra chromosomal element often carrying genes which are not part of the central metabolism of the cell, and usually in the form of circular double-stranded DNA molecules. Such elements may be autonomously replicating sequences, genome integrating sequences, phage or nucleotide sequences, linear or circular, of a single- or double-stranded DNA or RNA, derived from any source, in which a number of nucleotide sequences have been joined or recombined into a unique construction which is capable of introducing a promoter fragment and DNA sequence for a selected gene product along with appropriate 3′ untranslated sequence into a cell. “Transformation cassette” refers to a specific vector containing a foreign gene and having elements in addition to the foreign gene that facilitate transformation of a particular host cell. “Expression cassette” refers to a specific vector containing a foreign gene and having elements in addition to the foreign gene that allow for enhanced expression of that gene in a foreign host.

The term “phage” or “bacteriophage” refers to a virus that infects bacteria. Altered forms may be used for the purpose of the present invention. The preferred bacteriophage is derived from the “wild” phage, called M13. The M13 system can grow inside a bacterium, so that it does not destroy the cell it infects but causes it to make new phages continuously. It is a single-stranded DNA phage.

The term “phage display” refers to the display of functional foreign peptides or small proteins on the surface of bacteriophage or phagemid particles. Genetically engineered phage may be used to present peptides as segments of their native surface proteins. Peptide libraries may be produced by populations of phage with different gene sequences.

“PCR” or “polymerase chain reaction” is a technique used for the amplification of specific DNA segments (U.S. Pat. Nos. 4,683,195 and 4,800,159).

Standard recombinant DNA and molecular cloning techniques used herein are well known in the art and are described by Sambrook, J., Fritsch, E. F. and Maniatis, T., Molecular Cloning: A Laboratory Manual, Second Edition, Cold Spring Harbor Laboratory Press, Cold Spring Harbor, N.Y. (1989) (hereinafter “Maniatis”); and by Silhavy, T. J., Bennan, M. L. and Enquist, L. W., Experiments with Gene Fusions, Cold Spring Harbor Laboratory Cold Press Spring Harbor, N.Y. (1984); and by Ausubel, F. M. et al., Current Protocols in Molecular Biology, published by Greene Publishing Assoc. and Wiley-Interscience (1987).

The invention provides a method for identifying hair conditioner-resistant peptide sequences that bind specifically to hair with high affinity in the presence of a hair conditioner matrix. The method is a modification of standard biopanning techniques wherein hair is contacted with a library of combinatorially generated peptides. Within the context of the present invention the resulting DNA associated peptide-hair complex is contacted with a hair conditioner matrix for a period of time. The DNA associated peptide-hair complex is isolated and optionally contacted with an eluting agent to give eluted DNA associated peptides and DNA associated peptides that remain bound to the hair. The eluted DNA associated peptides and/or the remaining bound DNA associated peptides are amplified and identified. The identified hair conditioner-resistant hair-binding peptide sequences may be used to construct peptide-based hair benefit agents, such as hair conditioners and colorants.

### Identification of Hair Conditioner-Resistant Hair-Binding Peptides

Hair conditioner-resistant hair-binding peptides (HCP), as defined herein, are peptide sequences that specifically bind to hair from a hair conditioner matrix and are stable therein. The hair conditioner-resistant hair-binding peptides of the invention are from about 7 amino acids to about 45 amino acids in length, more preferably, from about 7 amino acids to about 25 amino acids in length, most preferably from about 12 to about 20 amino acids in length. The peptides of the present invention are generated randomly and then selected against a hair sample based upon their binding affinity for the hair in the presence of a hair conditioner matrix, as described below.

The generation of random libraries of peptides is well known and may be accomplished by a variety of techniques including, bacterial display (Kemp, D. J.; Proc. Natl. Acad. Sci. USA 78(7):4520-4524 (1981), and Helfman et al., Proc. Natl. Acad. Sci. USA 80(1):31-35, (1983)), yeast display (Chien et al., Proc Natl Acad Sci USA 88(21):9578-82 (1991)), combinatorial solid phase peptide synthesis (U.S. Pat. No. 5,449,754, U.S. Pat. No. 5,480,971, U.S. Pat. No. 5,585,275, U.S. Pat. No. 5,639,603), and phage display technology (U.S. Pat. No. 5,223,409, U.S. Pat. No. 5,403,484, U.S. Pat. No. 5,571,698, U.S. Pat. No. 5,837,500). Techniques to generate such biological peptide libraries are well known in the art. Exemplary methods are described in Dani, M., J. of Receptor & Signal Transduction Res., 21(4):447-468 (2001), Sidhu et al., Methods in Enzymology 328:333-363 (2000), and Phage Display of Peptides and Proteins, A Laboratory Manual, Brian K. Kay, Jill Winter, and John McCafferty, eds.; Academic Press, NY, 1996. Additionally, phage display libraries may be purchased from commercial sources, such as New England Biolabs (Beverly, Mass.).

In one embodiment it is particularly useful to have the DNA encoding the peptide associated with the peptide in some manner. This association facilitates rapid identification of the binding peptide in the screening or biopanning process. The coding DNA may be either PCR amplified or used to infect a replicating host to increase the expression of the peptide for facile identification. Typically DNA associated peptides are produced by the methods of phage display, bacteria display and yeast display as referenced above.

A preferred method to randomly generate peptides is by phage display. Phage display is an in vitro selection technique in which a peptide or protein is genetically fused to a coat protein of a bacteriophage, resulting in display of fused peptide on the exterior of the phage virion, while the DNA encoding the fusion resides within the virion. This physical linkage between the displayed peptide and the DNA encoding it allows screening of vast numbers of variants of peptides, each linked to a corresponding DNA sequence, by a simple in vitro selection procedure called “biopanning”. In its simplest form, biopanning is carried out by incubating the pool of phage-displayed variants with a target of interest, washing away unbound phage, and eluting specifically bound phage by disrupting the binding interactions between the phage and the target. The eluted phage is then amplified in vivo and the process is repeated, resulting in a stepwise enrichment of the phage pool in favor of the tightest binding sequences. After 3 or more rounds of selection/amplification, individual clones are characterized by DNA sequencing.

The hair conditioner-resistant hair-binding peptides of the invention may be identified using phage display by selecting phage peptides against a hair sample based upon their binding affinity for the hair in the presence of a hair conditioner matrix. The hair and the phage peptides may be contacted with the hair conditioner matrix in various ways to form a conditioning solution, as described in detail below. For example, the phage peptide library may be dissolved in the hair conditioner matrix which is then contacted with the hair sample. Alternatively, the phage-peptide-hair complex, formed by contacting the hair sample with the phage display library, may be subsequently contacted with a hair conditioner matrix. Additionally, any combination of these hair conditioner-contacting methods may be used.

After a suitable library of DNA associated peptides has been generated or purchased from a commercial supplier, the library is contacted with an appropriate amount of hair sample to form a reaction solution comprising DNA associated peptide-hair complexes. Human hair samples are available commercially, for example from International Hair Importers and Products (Bellerose, N.Y.), in different colors, such as brown, black, red, and blond, and in various types, such as African-American, Caucasian, and Asian. Additionally, the hair samples may be treated, for example using hydrogen peroxide to obtain bleached hair. The library of DNA associated peptides is dissolved in a suitable solution for contacting the hair sample. In one embodiment, the library of peptides is dissolved in a buffered aqueous saline solution containing a surfactant. A suitable solution is Tris-buffered saline (TBS) with 0.5% Tween® 20. In another embodiment, the library of peptides is dissolved in a hair conditioner matrix (see below) and then contacted with the hair sample. The solution containing the peptide library may be agitated by any means in order to increase the mass transfer rate of the peptides to the hair surface, thereby shortening the time required to attain maximum binding. The time required to attain maximum binding varies depending on a number of factors, such as size of the hair sample, the concentration of the peptide library, and the agitation rate. The time required can be determined readily by one skilled in the art using routine experimentation. Typically, the contact time is one minute to one hour. Optionally, the library of peptides may be contacted with a non-target, such as skin or plastic, either prior to or simultaneously with contacting the hair sample to remove the undesired DNA associated peptides that bind to the non-target.

Upon contact with the hair sample, a number of the randomly generated peptides bind to the hair to form DNA associated peptide-hair complexes. A number of peptides remain uncomplexed and portions of the hair sample are also unbound. Uncomplexed peptides may optionally be removed by washing using any suitable buffer solution, such as Tris-HCl, Tris-buffered saline, Tris-borate, Tris-acetic acid, triethylamine, phosphate buffer, and glycine-HCl, wherein Tris-buffered saline solution is preferred. The wash solution may also contain a surfactant such as SDS (sodium dodecyl sulfate), DOC (sodium deoxycholate), Nonidet P-40, Triton X-100, and Tween® 20, wherein Tween® 20 at a concentration of 0.5% is preferred. The wash step may be repeated one or more times.

After the uncomplexed material is removed, the DNA associated peptide-hair complexes are contacted with a hair conditioner matrix for a period of time, typically, about 1 minute to about 30 minutes, to form a conditioning solution. A hair conditioner matrix, as used herein, refers to a medium comprising a hair conditioner product, either undiluted or in diluted form, or a mixture comprising at least one component, in addition, at least two components of a hair conditioner product. Suitable hair conditioner product compositions are well-known in the art. Components of hair conditioner product compositions are described by Philippe et al. in U.S. Pat. No. 6,280,747, and by Omura et al. in U.S. Pat. No. 6,139,851 and Cannell et al. in U.S. Pat. No. 6,013,250, all of which are incorporated herein by reference. For example, the hair conditioner composition can be an aqueous solution, an aqueous-alcoholic solution, and a water-in-oil (W/O) or an oil-in-water (O/W) emulsion. Additionally, the hair conditioner composition may contain one or more conventional cosmetic or dermatological additives or adjuvants including but not limited to, hair conditioning agents (see below for examples), antioxidants, preserving agents, fillers, surfactants, UVA and/or UVB sunscreens, fragrances, thickeners, wetting agents and anionic, nonionic or amphoteric polymers, and dyes or pigments. These adjuvants are well known in the field of cosmetics and are described in many publications, for example see Harry's Book of Cosmeticology, 8^{th} edition, Martin Rieger, ed., Chemical Publishing, New York (2000). Additionally, commercially available hair conditioner products, such as Dove® Extra Volume Conditioner (Unilever), Pantene Pro V (Proctor and Gamble), Herbal Essence (Clairol), Finesse (Helene Curtis), and Tresemme (Alberto Culver) may be used. Hair conditioners may be purchased at local supermarkets and pharmacies. Preferably, the hair conditioner matrix in which the hair conditioner-resistant hair-binding peptide will ultimately be employed, is used in the method. The hair conditioner composition may be used undiluted or may be diluted to facilitate its application, particularly in the case of a very viscous composition. The hair conditioner matrix may be diluted with water or a suitable buffer solution, such as that described above, may be used. The concentration of the hair conditioner matrix is at least about 10%, preferably at least about 20%, more preferably at least about 50%, and more preferably at least about 75% of full strength concentration. Most preferably, the hair conditioner matrix is used in undiluted form. Optionally the DNA associated peptide-hair complex may be contacted with the hair conditioner matrix one or more times.

The DNA associated peptide-hair complexes are isolated from the conditioning solution and are optionally washed one or more times using a buffer solution, as described above. The hair conditioner matrix may also be used as the wash solution. The DNA associated peptide-hair complexes are then contacted with an eluting agent, preferably after being transferred to a new container, to dissociate the DNA associated peptides from the hair; however, some of the DNA associated peptides may still remain bound to the hair after this treatment. The eluting agent may be any known eluting agent including, but not limited to, acid (pH 1.5-3.0); base (pH 10-12.5); salt solutions containing high salt concentrations such as MgCl₂ (3-5 M) and LiCl (5-10 M); water; ethylene glycol (25-50%); dioxane (5-20%); thiocyanate (1-5 M); guanidine (2-5 M); and urea (2-8 M), wherein treatment with an acid is preferred. If the elution buffer used is an acid or base, then, a neutralization buffer is added to adjust the pH to the neutral range after the elution step. Any suitable buffer may be used, wherein 1 M Tris-HCl pH 9.2 is preferred for use with an acid elution buffer.

The DNA encoding the eluted peptides or the remaining bound peptides, or the DNA encoding both the eluted peptides and the remaining bound peptides is then amplified using methods known in the art. For example, the DNA encoding the eluted peptides and the remaining bound peptides may be amplified by infecting a bacterial host cell, such as E. coli ER2738, with the DNA encoding the desired peptide, as described by Huang et al. (copending and commonly owned U.S. Patent Application Publication No. 2005/0050656, incorporated herein by reference). The infected host cells are grown in an appropriate growth medium, such as LB (Luria-Bertani) medium, and this culture is spread onto agar, containing a suitable growth medium, such as LB medium with IPTG (isopropyl β-D-thiogalactopyranoside) and S-Gal™ (3,4-cyclohexenoesculetin-β-D-galactopyranoside). After growth, the plaques are picked for DNA isolation and sequencing to identify sequences encoding the hair conditioner-resistant hair-binding peptide sequences. Alternatively, the DNA encoding the eluted peptides and the remaining bound peptides may be amplified using a nucleic acid amplification method, such as the polymerase chain reaction (PCR). In that approach, PCR is carried out on the DNA encoding the eluted peptides and/or the remaining bound peptides using the appropriate primers, as described by Janssen et al. in U.S. Patent Application Publication No. 2003/0152976, which is incorporated herein by reference.

In one embodiment, the DNA encoding the eluted peptides and the remaining bound peptides are amplified by infecting a bacterial host cell, the amplified DNA associated peptides are contacted with a fresh hair sample, and the entire process described above is repeated one or more times to obtain a population that is enriched in hair conditioner-resistant hair-binding DNA associated peptides. After the desired number of biopanning cycles, the amplified DNA sequences are determined using standard DNA sequencing techniques that are well known in the art to identify the hair conditioner-resistant hair-binding peptide sequences.

Hair conditioner-resistant hair-binding peptides have been identified using the above methods. Specifically, binding peptides, given as SEQ ID NOs:1-5, were isolated that have a high affinity for normal brown hair from a hair conditioner matrix and are stable therein.

### Production of Hair Conditioner-Resistant Hair-Binding Peptides

The hair conditioner-resistant hair-binding peptides of the present invention may be prepared using standard peptide synthesis methods, which are well known in the art (see for example Stewart et al., Solid Phase Peptide Synthesis, Pierce Chemical Co., Rockford, Ill., 1984; Bodanszky, Principles of Peptide Synthesis, Springer-Verlag, New York, 1984; and Pennington et al., Peptide Synthesis Protocols, Humana Press, Totowa, N.J., 1994). Additionally, many companies offer custom peptide synthesis services.

Alternatively, the peptides of the present invention may be prepared using recombinant DNA and molecular cloning techniques. Genes encoding the hair-binding peptides may be produced in heterologous host cells, particularly in the cells of microbial hosts.

Preferred heterologous host cells for expression of the binding peptides of the present invention are microbial hosts that can be found broadly within the fungal or bacterial families and which grow over a wide range of temperature, pH values, and solvent tolerances. Because transcription, translation, and the protein biosynthetic apparatus are the same irrespective of the cellular feedstock, functional genes are expressed irrespective of carbon feedstock used to generate cellular biomass. Examples of host strains include, but are not limited to, fungal or yeast species such as Aspergillus, Trichoderma, Saccharomyces, Pichia, Candida, Hansenula, or bacterial species such as Salmonella, Bacillus, Acinetobacter, Rhodococcus, Streptomyces, Escherichia, Pseudomonas, Methylomonas, Methylobacter, Alcaligenes, Synechocystis, Anabaena, Thiobacillus, Methanobacterium and Klebsiella.

A variety of expression systems can be used to produce the peptides of the present invention. Such vectors include, but are not limited to, chromosomal, episomal and virus-derived vectors, e.g., vectors derived from bacterial plasmids, from bacteriophage, from transposons, from insertion elements, from yeast episoms, from viruses such as baculoviruses, retroviruses and vectors derived from combinations thereof such as those derived from plasmid and bacteriophage genetic elements, such as cosmids and phagemids. The expression system constructs may contain regulatory regions that regulate as well as engender expression. In general, any system or vector suitable to maintain, propagate or express polynucleotide or polypeptide in a host cell may be used for expression in this regard. Microbial expression systems and expression vectors contain regulatory sequences that direct high level expression of foreign proteins relative to the growth of the host cell. Regulatory sequences are well known to those skilled in the art and examples include, but are not limited to, those which cause the expression of a gene to be turned on or off in response to a chemical or physical stimulus, including the presence of regulatory elements in the vector, for example, enhancer sequences. Any of these could be used to construct chimeric genes for production of the any of the binding peptides of the present invention. These chimeric genes could then be introduced into appropriate microorganisms via transformation to provide high level expression of the peptides.

Vectors or cassettes useful for the transformation of suitable host cells are well known in the art. Typically the vector or cassette contains sequences directing transcription and translation of the relevant gene, one or more selectable markers, and sequences allowing autonomous replication or chromosomal integration. Suitable vectors comprise a region 5′ of the gene, which harbors transcriptional initiation controls and a region 3′ of the DNA fragment which controls transcriptional termination. It is most preferred when both control regions are derived from genes homologous to the transformed host cell, although it is to be understood that such control regions need not be derived from the genes native to the specific species chosen as a production host. Selectable marker genes provide a phenotypic trait for selection of the transformed host cells such as tetracycline or ampicillin resistance in E. coli.

Initiation control regions or promoters which are useful to drive expression of the chimeric gene in the desired host cell are numerous and familiar to those skilled in the art. Virtually any promoter capable of driving the gene is suitable for producing the binding peptides of the present invention including, but not limited to: CYC1, HIS3, GAL1, GAL10, ADH1, PGK, PHO5, GAPDH, ADC1, TRP1, URA3, LEU2, ENO, TPI (useful for expression in Saccharomyces); AOX1 (useful for expression in Pichia); and lac, ara, tet, trp, IP_{L}, IP_{R}, T7, tac, and trc (useful for expression in Escherichia coli) as well as the amy, apr, npr promoters and various phage promoters useful for expression in Bacillus.

Termination control regions may also be derived from various genes native to the preferred hosts. Optionally, a termination site may be unnecessary, however, it is most preferred if included.

The vector containing the appropriate DNA sequence as described supra, as well as an appropriate promoter or control sequence, may be employed to transform an appropriate host to permit the host to express the peptide of the present invention. Cell-free translation systems can also be employed to produce such peptides using RNAs derived from the DNA constructs of the present invention. Optionally it may be desired to produce the instant gene product as a secretion product of the transformed host. Secretion of desired proteins into the growth media has the advantages of simplified and less costly purification procedures. It is well known in the art that secretion signal sequences are often useful in facilitating the active transport of expressible proteins across cell membranes. The creation of a transformed host capable of secretion may be accomplished by the incorporation of a DNA sequence that codes for a secretion signal which is functional in the production host. Methods for choosing appropriate signal sequences are well known in the art (see for example EP 546049 and WO 9324631). The secretion signal DNA or facilitator may be located between the expression-controlling DNA and the instant gene or gene fragment, and in the same reading frame with the latter.

### Peptide-Based Hair Benefit Agents

The peptide-based hair benefit agents of the invention are formed by coupling a hair conditioner-resistant hair-binding peptide (HCP) with a benefit agent (BA), such as a conditioner, colorant, fragrance, sunscreen, and the like. The hair conditioner-resistant hair-binding peptide part of the peptide-based benefit agent binds strongly to the hair from a hair conditioner matrix, thus keeping the benefit agent attached to the hair for a long lasting effect. The coupling interaction between the hair conditioner-resistant hair-binding peptide and the benefit agent may be a covalent bond or a non-covalent interaction and may be through an optional spacer, as described below.

It may also be desirable to have multiple hair conditioner-resistant hair-binding peptides coupled to the benefit agent to enhance the interaction between the peptide-based benefit agent and the hair, as described by Huang et al., (copending and commonly owned U.S. Patent Application Publication No. 2005/0050656). This may be done by coupling multiple copies of single hair conditioner-resistant hair-binding sequences to the benefit agent or by linking two or more hair conditioner-resistant hair-binding peptide sequences together, either directly or through a spacer, and coupling the resulting multi-copy hair-binding sequence to the benefit agent. Additionally, multiple copies of the multi-copy hair conditioner-resistant hair-binding peptide sequence may be coupled to the benefit agent. In all these peptide-based hair benefit agents, multiple copies of the same hair conditioner-resistant hair-binding peptide or a combination of different hair conditioner-resistant hair-binding peptides may be used.

In one embodiment of the present invention, the peptide-based benefit agents are diblock compositions consisting of a hair conditioner-resistant hair-binding peptide (HCP) and a benefit agent (BA), having the general structure (HCPₘ)ₙ—BA, where m ranges from 1 to about 100, preferably from 1 to about 10. When the benefit agent is a molecular species, n ranges from 1 to about 100, preferably from 1 to about 10. When the benefit agent is a particle, such as a pigment, n ranges from 1 to about 50,000, preferably from 1 to about 10,000.

In another embodiment, the peptide-based benefit agents contain a spacer (S) separating the hair conditioner-resistant hair-binding peptide from the benefit agent. Multiple copies of the hair conditioner-resistant hair-binding peptide may be coupled to a single spacer molecule. Alternatively, multiple copies of hair conditioner-resistant hair-binding peptides may be separated by various spacers. In this embodiment, the peptide-based benefit agents are triblock compositions consisting of a hair conditioner-resistant hair-binding peptide, a spacer, and a benefit agent, having the general structure [(HCP_{X}—S)ₘ]ₙ—BA, where x ranges from 1 to about 10, preferably x is 1, and m ranges from 1 to about 100, preferably from 1 to about 10. When the benefit agent is a molecular species, such as a dye or non-particle conditioning agent, n ranges from 1 to about 100, preferably from 1 to about 10. When the benefit agent is a particle, such as a pigment, n ranges from 1 to about 50,000, preferably from 1 to about 10,000.

It should be understood that as used herein, HCP is a generic designation and is not meant to refer to a single hair conditioner-resistant hair-binding peptide sequence. Where m, n or x, as used above, is greater than 1, it is well within the scope of the invention to provide for the situation where a series of hair binding peptides of different sequences may form a part of the composition. Additionally, S is a generic designation and is not meant to refer to a single spacer. Where m or n, as used above for the triblock compositions, is greater than 1, it is well within the scope of the invention to provide for the situation where a series of different spacers may form a part of the composition. It should also be understood that these structures do not necessarily represent a covalent bond between the peptide, the benefit agent, and the optional spacer. As described below, the coupling interaction between the peptide, the benefit agent, and the optional spacer may be either covalent or non-covalent.

The preparation of the hair conditioner-resistant peptide-based benefit agents of the invention is described below for hair conditioner and hair colorants. It should be understood that these methods may be applied to other benefit agents and that these other hair conditioner-resistant peptide-based benefit agents are within the scope of the invention.

### Peptide-Based Hair Conditioners

The peptide-based hair conditioners of the invention are formed by coupling a hair conditioner-resistant hair-binding peptide (HCP) with a hair conditioning agent (HCA). The hair conditioner-resistant hair-binding peptide part of the conditioner binds strongly to the hair from a hair conditioner matrix, thus keeping the conditioning agent attached to the hair for a long lasting conditioning effect. The hair conditioner-resistant hair-binding peptides are selected by the methods described above, and include, but are not limited to, the hair-binding peptide sequences given as SEQ ID NOs:1-5, and SEQ ID NO:12.

Hair conditioning agents as herein defined are agents which improve the appearance, texture, and sheen of hair as well as increasing hair body or suppleness. Hair conditioning agents, include, but are not limited to, styling aids, hair straightening aids, hair strengthening aids, and volumizing agents, such as nanoparticles. In the peptide-based hair conditioners of the present invention, any suitable hair conditioning agent may be used. Hair conditioning agents are well known in the art, see for example Green et al. (WO 0107009), incorporated herein by reference, and are available commercially from various sources. Suitable examples of hair conditioning agents include, but are not limited to, cationic polymers, such as cationized guar gum, diallyly quaternary ammonium salt/acrylamide copolymers, quaternized polyvinylpyrrolidone and derivatives thereof, and various polyquaternium-compounds; cationic surfactants, such as stearalkonium chloride, centrimonium chloride, and Sapamin hydrochloride; fatty alcohols, such as behenyl alcohol; fatty amines, such as stearyl amine; waxes; esters; nonionic polymers, such as polyvinylpyrrolidone, polyvinyl alcohol, and polyethylene glycol; silicones; siloxanes, such as decamethylcyclopentasiloxane; polymer emulsions, such as amodimethicone; and nanoparticles, such as silica nanoparticles and polymer nanoparticles. The preferred hair conditioning agents of the present invention contain amine or hydroxyl functional groups to facilitate coupling to the hair-binding peptides, as described below. Examples of preferred conditioning agents are octylamine (CAS No. 111-86-4), stearyl amine (CAS No. 124-30-1), behenyl alcohol (CAS No. 661-19-8, Cognis Corp., Cincinnati, Ohio), vinyl group terminated siloxanes, vinyl group terminated silicone (CAS No. 68083-19-2), vinyl group terminated methyl vinyl siloxanes, vinyl group terminated methyl vinyl silicone (CAS No. 68951-99-5), hydroxyl terminated siloxanes, hydroxyl terminated silicone (CAS No. 80801-30-5), amino-modified silicone derivatives, [(aminoethyl)amino]propyl hydroxyl dimethyl siloxanes, [(aminoethyl)amino]propyl hydroxyl dimethyl silicones, and alpha-tridecyl-omega-hydroxy-poly(oxy-1,2-ethanediyl) (CAS No. 24938-91-8).

The peptide-based hair conditioners of the present invention are prepared by coupling a specific hair conditioner-resistant hair-binding peptide to a hair conditioning agent, either directly or via an optional spacer. The coupling interaction may be a covalent bond or a non-covalent interaction, such as hydrogen bonding, electrostatic interaction, hydrophobic interaction, or Van der Waals interaction. In the case of a non-covalent interaction, the peptide-based hair conditioner may be prepared by mixing the peptide with the conditioning agent and the optional spacer (if used) and allowing sufficient time for the interaction to occur. The unbound materials may be separated from the resulting peptide-based hair conditioner adduct using methods known in the art, for example, gel permeation chromatography.

The peptide-based hair conditioners of the invention may also be prepared by covalently attaching a specific hair conditioner-resistant hair-binding peptide to a hair conditioning agent, either directly or through a spacer, as described by Huang et al. (copending and commonly owned U.S. Patent Application Publication No. 2005/0050656). Any suitable known peptide or protein conjugation chemistry may be used to form the peptide-based hair conditioners of the present invention. Conjugation chemistries are well-known in the art (see for example, Hermanson, Bioconjugate Techniques, Academic Press, New York (1996)). Suitable coupling agents include, but are not limited to, carbodiimide coupling agents, acid chlorides, isocyanates, epoxides, maleimides, and other functional coupling reagents that are reactive toward terminal amine and/or carboxylic acid groups, and sulfhydryl groups on the peptides. Additionally, it may be necessary to protect reactive amine or carboxylic acid groups on the peptide to produce the desired structure for the peptide-based conditioner. The use of protecting groups for amino acids, such as t-butyloxycarbonyl (t-Boc), are well known in the art (see for example Stewart et al., supra; Bodanszky, supra; and Pennington et al., supra). In some cases it may be necessary to introduce reactive groups, such as carboxylic acid, alcohol, amine, isocyanate, or aldehyde groups on the conditioning agent for coupling to the hair-binding peptide. These modifications may be done using routine chemistry such as oxidation, reduction, phosgenation, and the like, which is well known in the art.

It may also be desirable to couple the hair conditioner-resistant hair-binding peptide to the hair conditioning agent via a spacer. The spacer serves to separate the conditioning agent from the peptide to ensure that the agent does not interfere with the binding of the peptide to the hair. The spacer may be any of a variety of molecules, such as alkyl chains, phenyl compounds, ethylene glycol, amides, esters and the like. Preferred spacers are hydrophilic and have a chain length from 1 to about 100 atoms, more preferably, from 2 to about 30 atoms. Examples of preferred spacers include, but are not limited to, ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, and ethyl, propyl, hexyl, steryl, cetyl, and palmitoyl alkyl chains. The spacer may be covalently attached to the peptide and the hair conditioning agent using any of the coupling chemistries described above. In order to facilitate incorporation of the spacer, a bifunctional cross-linking agent that contains a spacer and reactive groups at both ends for coupling to the peptide and the conditioning agent may be used. Suitable bifunctional cross-linking agents are well known in the art and include, but are not limited to diamines, such a as 1,6-diaminohexane; dialdehydes, such as glutaraldehyde; bis N-hydroxysuccinimide esters, such as ethylene glycol-bis(succinic acid N-hydroxysuccinimide ester), disuccinimidyl glutarate, disuccinimidyl suberate, and ethylene glycol-bis(succinimidylsuccinate); diisocyantes, such as hexamethylenediisocyanate; bis oxiranes, such as 1,4 butanediyl diglycidyl ether; dicarboxylic acids, such as succinyldisalicylate; and the like. Heterobifunctional cross-linking agents, which contain a different reactive group at each end, may also be used. Examples of heterobifunctional cross-linking agents include, but are not limited to compounds having the following structure:

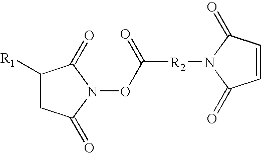

where: R₁ is H or a substituent group such as —SO₃Na, —NO₂, or —Br; and R₂ is a spacer such as —CH₂CH₂ (ethyl), —(CH₂)₃ (propyl), or —(CH₂)₃C₆H₅ (propyl phenyl). An example of such a heterobifunctional cross-linking agent is 3-maleimidopropionic acid N-hydroxysuccinimide ester. The N-hydroxysuccinimide ester group of these reagents reacts with amine or alcohol groups on the conditioner, while the maleimide group reacts with thiol groups present on the peptide. A thiol group may be incorporated into the peptide by adding at least one cysteine residue to at least one end of the binding peptide sequence, i.e., the C-terminal end or the N-terminal end. Several spacer amino acid residues, such as glycine, may be incorporated between the binding peptide sequence and the terminal cysteine to separate the reacting thiol group from the binding sequence. Moreover, at least one lysine residue may be added to at least one end of the binding peptide sequence, i.e., the C-terminal end or the N-terminal end, to provide an amine group for coupling.

Additionally, the spacer may be a peptide comprising any amino acid and mixtures thereof. The preferred peptide spacers comprise the amino acids proline, lysine, glycine, alanine, and serine, and mixtures thereof. In addition, the peptide spacer may comprise a specific enzyme cleavage site, such as the protease Caspase 3 site, given by SEQ ID NO:6, which allows for the enzymatic removal of the conditioning agent from the hair. The peptide spacer may be from 1 to about 50 amino acids, preferably from 1 to about 20 amino acids in length. Examples of peptide spacers include, but are not limited to, SEQ ID NOs:13-15. These peptide spacers may be linked to the binding peptide sequence by any method know in the art. For example, the entire binding peptide-peptide spacer-diblock may be prepared using the standard peptide synthesis methods described supra. In addition, the binding peptide and peptide spacer blocks may be combined using carbodiimide coupling agents (see for example, Hermanson, Bioconjugate Techniques, Academic Press, New York (1996)), diacid chlorides, diisocyanates and other difunctional coupling reagents that are reactive to terminal amine and/or carboxylic acid groups on the peptides. Alternatively, the entire binding peptide-peptide spacer-diblock may be prepared using the recombinant DNA and molecular cloning techniques described supra. The spacer may also be a combination of a peptide spacer and an organic spacer molecule, which may be prepared using the methods described above.

It may also be desirable to have multiple hair conditioner-resistant hair-binding peptides coupled to the hair conditioning agent to enhance the interaction between the peptide-based hair conditioner and the hair. Either multiple copies of the same hair-binding peptide or a combination of different hair-binding peptides may be used. For example, a combination of hair conditioner-resistant and shampoo-resistant hair-binding peptides may be used. Shampoo resistant hair-binding peptides are described by Huang et al. (copending and commonly owned U.S. Patent Application Publication No. 2005/0050656) and by O'Brien et al. (copending and commonly owned U.S. patent application Ser. No. 11/251,715). The multi-copy hair conditioner-resistant hair binding peptides may comprise various spacers, as described above. In the case of large conditioning particles (e.g., particle emulsions or nanoparticles), a large number of hair-binding peptides, i.e., up to about 50,000, may be coupled to the conditioning agent. A smaller number of hair-binding peptides can be coupled to the smaller conditioner molecules, i.e., up to about 100. Additionally, multiple hair-binding peptide sequences may be linked together and attached to the conditioning agent. Therefore, in one embodiment of the present invention, the peptide-based hair conditioners are diblock compositions consisting of a hair conditioner-resistant hair-binding peptide (HCP) and a hair conditioning agent (HCA), having the general structure (HCPₘ)ₙ—HCA, where m ranges from 1 to about 100, preferably from 1 to about 10. When the hair conditioning agent is a molecular species, i.e., a non-particle conditioning agent, n ranges from 1 to about 100, preferably from 1 to about 10. When the hair conditioning agent is a particle, n ranges from 1 to about 50,000, preferably from 1 to about 10,000.

In another embodiment, the peptide-based hair conditioners contain a spacer (S) separating the hair conditioner-resistant hair-binding peptide from the hair conditioning agent, as described above. Multiple copies of the hair-binding peptide may be coupled to a single spacer molecule. Additionally, multiple copies of the peptides may be linked together via spacers and coupled to the hair conditioning agent via a spacer. In this embodiment, the peptide-based hair conditioners are triblock compositions consisting of a hair conditioner-resistant hair-binding peptide, a spacer, and a hair conditioning agent, having the general structure [(HCP_{X}—S)ₘ]ₙ—HCA, where x ranges from 1 to about 10, preferably x is 1, and m ranges from 1 to about 100, preferably from 1 to about 10. When the hair conditioning agent is a molecular species, i.e., a non-particle conditioning agent, n ranges from 1 to about 100, preferably from 1 to about 10. When the hair conditioning agent is a particle, n ranges from 1 to about 50,000, preferably from 1 to about 10,000.

It should be understood that as used herein, HCP is a generic designation and is not meant to refer to a single hair-binding peptide sequence. Where m, n or x as used above, is greater than 1, it is well within the scope of the invention to provide for the situation where a series of hair binding peptides of different sequences may form a part of the composition. Additionally, S is a generic designation and is not meant to refer to a single spacer. Where m or n, as used above for the triblock compositions, is greater than 1, it is well within the scope of the invention to provide for the situation where a series of different spacers may form a part of the composition. It should also be understood that these structures do not necessarily represent a covalent bond between the peptide, the hair conditioning agent, and the optional spacer. As described above, the coupling interaction between the peptide, the hair conditioning agent, and the optional spacer may be either covalent or non-covalent.

The peptide-based hair conditioners of the present invention may be used in products for hair care. It should also be recognized that the hair conditioner-resistant hair-binding peptides themselves can serve as conditioning agents for the treatment of hair. Hair care product compositions are herein defined as compositions for the treatment of hair, including but not limited to, conditioners, lotions, aerosols, gels, mousses, and hair colorants. In one embodiment, the hair care product composition is a hair conditioning product. In another embodiment, the hair care product composition is a hair coloring product.

The hair care product compositions of the invention comprise an effective amount of a peptide-based hair conditioner or a mixture of different peptide-based hair conditioners in a cosmetically acceptable medium. An effective amount of a peptide-based hair conditioner or hair-binding peptide for use in a hair care product composition is herein defined as a proportion of from about 0.01% to about 10%, preferably about 0.01% to about 5% by weight relative to the total weight of the composition. Components of a cosmetically acceptable medium for hair care product compositions are well-known in the art and examples are described by Philippe et al. in U.S. Pat. No. 6,280,747, Omura et al. in U.S. Pat. No. 6,139,851 and Cannell et al. in U.S. Pat. No. 6,013,250.

### Peptide-Based Hair Colorants

The peptide-based hair colorants of the invention are formed by coupling a hair-conditioner-resistant hair-binding peptide (HCP) with a coloring agent (C). The hair conditioner-resistant hair-binding peptide part of the peptide-based hair colorant binds strongly to the hair and is not removed by the application of a hair conditioner, thus keeping the coloring agent attached to the hair for a long lasting hair coloring effect. The hair conditioner-resistant hair-binding peptides are selected by the methods described and include, but are not limited to, the hair-binding peptide sequences given as SEQ ID NOs:1-5, and SEQ ID NO:12.

Coloring agents as herein defined are any dye, pigment, and the like that may be used to change the color of hair. In the peptide-based hair colorants of the present invention, any suitable coloring agent may be used. Hair coloring agents are well known in the art (see for example Green et al. supra, CFTA International Color Handbook, 2^{nd} ed., Micelle Press, England (1992) and Cosmetic Handbook, US Food and Drug Administration, FDA/IAS Booklet (1992)), and are available commercially from various sources (for example Bayer, Pittsburgh, Pa.; Ciba-Geigy, Tarrytown, N.Y.; ICI, Bridgewater, N.J.; Sandoz, Vienna, Austria; BASF, Mount Olive, N.J.; and Hoechst, Frankfurt, Germany). Suitable hair coloring agents include, but are not limited to dyes, such as 4-hydroxypropylamino-3-nitrophenol, 4-amino-3-nitrophenol, 2-amino-6-chloro-4-nitrophenol, 2-nitro-paraphenylenediamine, N,N-hydroxyethyl-2-nitro-phenylenediamine, 4-nitro-indole, Henna, HC Blue 1, HC Blue 2, HC Yellow 4, HC Red 3, HC Red 5, Disperse Violet 4, Disperse Black 9, HC Blue 7, HC Blue 12, HC Yellow 2, HC Yellow 6, HC Yellow 8, HC Yellow 12, HC Brown 2, D&C Yellow 1, D&C Yellow 3, D&C Blue 1, Disperse Blue 3, Disperse Violet 1, eosin derivatives such as D&C Red No. 21 and halogenated fluorescein derivatives such as D&C Red No. 27, D&C Red Orange No. 5 in combination with D&C Red No. 21 and D&C Orange No. 10; and pigments, such as D&C Red No. 36 and D&C Orange No. 17, the calcium lakes of D&C Red Nos. 7, 11, 31 and 34, the barium lake of D&C Red No. 12, the strontium lake of D&C Red No. 13, the aluminum lakes of FD&C Yellow No. 5, of FD&C Yellow No. 6, of D&C Red No. 27, of D&C Red No. 21, and of FD&C Blue No. 1, iron oxides, manganese violet, chromium oxide, titanium dioxide, zinc oxide, barium oxide, ultramarine blue, bismuth citrate, and carbon black particles. Carbon nanotubes may also be used as a black pigment for dyeing hair, as described by Huang et al. in copending and commonly owned U.S. Patent Application Publication Nos. 2005/0229334 and 2005/0229335, both of which are incorporated herein by reference. The preferred dyes and pigments of the present invention include D&C Yellow 1 and 3, HC Yellow 6 and 8, D&C Blue 1, HC Blue 1, HC Brown 2, HC Red 5, 2-nitro-paraphenylenediamine, N,N-hydroxyethyl-2-nitro-phenylenediamine, titanium dioxide, 4-nitro-indole, iron oxides, carbon black, and carbon nanotubes.

Metallic and semiconductor nanoparticles may also be used as hair coloring agents due to their strong emission of light (Vic et al. U.S. Patent Application Publication No. 2004/0010864). The metallic nanoparticles include, but are not limited to, particles of gold, silver, platinum, palladium, iridium, rhodium, osmium, iron, copper, cobalt, and alloys composed of these metals. An “alloy” is herein defined as a homogeneous mixture of two or more metals. The “semiconductor nanoparticles” include, but are not limited to, particles of cadmium selenide, cadmium sulfide, silver sulfide, cadmium sulfide, zinc oxide, zinc sulfide, zinc selenide, lead sulfide, gallium arsenide, silicon, tin oxide, iron oxide, and indium phosphide. The nanoparticles are stabilized and made water-soluble by the use of a suitable organic coating or monolayer. As used herein, monolayer-protected nanoparticles are one type of stabilized nanoparticle. Methods for the preparation of stabilized, water-soluble metal and semiconductor nanoparticles are known in the art, and are described by Huang et al. in copending U.S. Patent Application Publication No. 2004/0115345, which is incorporated herein by reference. The color of the nanoparticles depends on the size of the particles. Therefore, by controlling the size of the nanoparticles, different colors may be obtained. For example, ZnS-coated CdSe nanoparticles cover the entire visible spectrum over a particle size range of 2 to 6 nm. Specifically, CdSe nanoparticles with a core size of 2.3, 4.2, 4.8 and 5.5 nm emit light at the wavelength centered around 485, 565, 590, and 625 nm, respectively. Water-soluble nanoparticles of different sizes may be obtained from a broad size distribution of nanoparticles using the size fractionation method described by Huang et al. (U.S. Patent Application Publication No. 2004/0115345). The method described therein comprises the regulated addition of a water-miscible organic solvent to a solution of nanoparticles in the presence of an electrolyte. Increasing additions of the water-miscible organic solvent result in the precipitation of nanoparticles of decreasing size. The metallic and semiconductor nanoparticles may also serve as volumizing agents, as described above.

Additionally, organic and inorganic nanoparticles, having an attached, adsorbed, or absorbed dye, may be used as a hair coloring agent. For example, the hair coloring agent may be colored polymer nanoparticles. Exemplary polymer nanoparticles include, but are not limited to, microspheres comprised of materials such as polystyrene, polymethylmethacrylate, polyvinyltoluene, styrene/butadiene copolymer, and latex. For use in the invention, the microspheres have a diameter of about 10 nanometers to about 2 microns. The microspheres may be colored by coupling any suitable dye, such as those described above, to the microspheres. The dyes may be coupled to the surface of the microsphere or adsorbed within the porous structure of a porous microsphere. Suitable microspheres, including undyed and dyed microspheres that are functionalized to enable covalent attachment, are available from companies such as Bang Laboratories (Fishers, Ind.).

The peptide-based hair colorants of the present invention are prepared by coupling a specific hair conditioner-resistant hair-binding peptide to a coloring agent, either directly or via a spacer. Any of the coupling methods described above may be used. It may be necessary to introduce reactive groups, such as carboxylic acid, alcohol, amine, aldehyde or isocyanate groups on the coloring agent for coupling to the hair-binding peptide. These modifications may be done using routine chemistry, such as oxidation, reduction, and phosgenation, which is well known in the art. For example, the surface of carbon black particles may be oxidized using nitric acid, a peroxide such as hydrogen peroxide, or an inorganic initiator such as ammonium persulfate, to generate functional groups. Preferably, the carbon black surface is oxidized using ammonium persulfate as described by Carrasco-Marin et al. (J. Chem. Soc., Faraday Trans. 93:2211-2215 (1997)). Amino functional groups may be introduced to the surface of carbon black using an organic initiator such as 2,2′-Azobis(2-methylpropionamide)-dihydrochloride. Inorganic pigments and nanoparticles may be derivatized to introduce carboxylic acid or amino functional groups in a similar manner.

It may also be desirable to have multiple hair conditioner-resistant hair-binding peptides coupled to the coloring agent to enhance the interaction between the peptide-based hair colorant and the hair. Either multiple copies of the same hair-binding peptide or a combination of different hair-binding peptides may be used. For example, a combination of hair conditioner-resistant and shampoo-resistant hair-binding peptides may be used, as described above. In the case of large pigment particles, a large number of hair-binding peptides, i.e., up to about 50,000, may be coupled to the pigment. A smaller number of hair-binding peptides can be coupled to the smaller dye molecules, i.e., up to about 100. Additionally, multiple hair-binding peptide sequences may be linked together and coupled to the coloring agent, as described above. Therefore, in one embodiment of the present invention, the peptide-based hair colorants are diblock compositions consisting of a hair conditioner-resistant hair-binding peptide (HCP) and a coloring agent (C), having the general structure (HCPₘ)ₙ—C, where m ranges from 1 to about 100, preferably m is 1 to about 10. When the coloring agent is a molecular species, such as a dye, n ranges from 1 to about 100, preferably from 1 to about 10. When the coloring agent is a particle, such as a pigment or nanoparticle, n ranges from 1 to about 50,000, preferably from 1 to about 10,000.

In another embodiment, the peptide-based hair colorants contain a spacer (S) separating the binding peptide from the hair coloring agent, as described above. Multiple copies of the hair conditioner-resistant hair-binding peptide may be coupled to a single spacer molecule. Additionally, multiple copies of the peptides may be linked together via spacers and coupled to the coloring agent via a spacer. In this embodiment, the peptide-based hair colorants are triblock compositions consisting of a hair conditioner-resistant hair-binding peptide, a spacer, and a coloring agent, having the general structure [(HCP_{X}—S)ₘ]ₙ—C, where x ranges from 1 to about 10, preferably x is 1, and m ranges from 1 to about 100, preferably m is 1 to about 10. When the coloring agent is a molecular species, such as a dye, n ranges from 1 to about 100, preferably from 1 to about 10. When the coloring agent is a particle, such as a pigment or nanoparticle, n ranges from 1 to about 50,000, preferably from 1 to about 10,000.

It should be understood that as used herein, HCP is a generic designation and is not meant to refer to a single hair-binding peptide sequence. Where m, n, or x, as used above, is greater than 1, it is well within the scope of the invention to provide for the situation where a series of hair binding peptides of different sequences may form a part of the composition. Additionally, S is a generic designation and is not meant to refer to a single spacer. Where m or n, as used above for the triblock compositions, is greater than 1, it is well within the scope of the invention to provide for the situation where a series of different spacers may form a part of the composition. It should also be understood that these structures do not necessarily represent a covalent bond between the peptide, the coloring agent, and the optional spacer. As described above, the coupling interaction between the peptide, the coloring agent, and the optional spacer may be either covalent or non-covalent.

The peptide-based hair colorants of the present invention may be used in hair coloring products for dyeing hair. Hair coloring product compositions are herein defined as compositions for the coloring, dyeing, or bleaching of hair, comprising an effective amount of peptide-based hair colorant or a mixture of different peptide-based hair colorants in a cosmetically acceptable medium. An effective amount of a peptide-based hair colorant for use in a hair coloring product composition is herein defined as a proportion of from about 0.001% to about 20% by weight relative to the total weight of the composition. Components of a cosmetically acceptable medium for hair coloring product compositions are described by Dias et al., in U.S. Pat. No. 6,398,821 and by Deutz et al., in U.S. Pat. No. 6,129,770, both of which are incorporated herein by reference. For example, hair coloring product compositions may contain sequestrants, stabilizers, thickeners, buffers, carriers, surfactants, solvents, antioxidants, polymers, and conditioners. The conditioners may include the peptide-based hair conditioners and hair conditioner-resistant hair-binding peptides of the present invention in a proportion from about 0.01% to about 10%, preferably about 0.01% to about 5% by weight relative to the total weight of the hair coloring composition.

The peptide-based hair colorants of the present invention may also be used as coloring agents in cosmetic product compositions that are applied to the eyelashes or eyebrows including, but not limited to, mascaras, and eyebrow pencils. These may be anhydrous make-up products comprising a cosmetically acceptable medium which contains a fatty substance in a proportion generally of from about 10 to about 90% by weight relative to the total weight of the composition, where the fatty phase containing at least one liquid, solid or semi-solid fatty substance. The fatty substance includes, but is not limited to, oils, waxes, gums, and so-called pasty fatty substances. Alternatively, these compositions may be in the form of a stable dispersion such as a water-in-oil or oil-in-water emulsion, as described above. In these compositions, the proportion of the peptide-based hair colorant is generally from about 0.001% to about 20% by weight relative to the total weight of the composition.

### Methods for Treating Hair

In another embodiment, methods are provided for treating hair with the peptide-based conditioners and colorants of the present invention. Specifically, the present invention also comprises a method for forming a protective film of peptide-based conditioner on hair by applying one of the compositions described above comprising an effective amount of a peptide-based hair conditioner to the hair and allowing the formation of the protective film. The compositions of the present invention may be applied to the hair by various means, including, but not limited to, spraying, brushing, and applying by hand. The peptide-based conditioner composition is left in contact with the hair for a period of time sufficient to form the protective film, preferably for at least about 0.1 to 60 min.

The present invention also provides a method for coloring hair by applying a hair coloring composition comprising an effective amount of a peptide-based hair colorant to the hair by means described above. The hair coloring composition is allowed to contact the hair for a period of time sufficient to cause coloration of the hair, preferably between about 5 to about 50 min, and then the hair coloring composition may be rinsed from the hair.

The present invention also provides a method for coloring eyebrows and eyelashes by applying a cosmetic composition comprising an effective amount of a peptide-based hair colorant to the eyebrows and eyelashes by means described above.

### EXAMPLES

The present invention is further defined in the following Examples. It should be understood that these Examples, while indicating preferred embodiments of the invention, are given by way of illustration only. From the above discussion and these Examples, one skilled in the art can ascertain the essential characteristics of this invention, and without departing from the spirit and scope thereof, can make various changes and modifications of the invention to adapt it to various uses and conditions.

The meaning of abbreviations used is as follows: “min” means minute(s), “sec” means second(s), “h” means hour(s), “μL” means microliter(s), “mL” means milliliter(s), “L” means liter(s), “nm” means nanometer(s), “mm” means millimeter(s), “cm” means centimeter(s), “μm” means micrometer(s), “mM” means millimolar, “M” means molar, “mmol” means millimole(s), “μmole” means micromole(s), “g” means gram(s), “μg” means microgram(s), “mg” means milligram(s), “pfu” means plague forming unit, “BSA” means bovine serum albumin, “ELISA” means enzyme linked immunosorbent assay, “A” means absorbance, “A450” means the absorbance measured at a wavelength of 450 nm, “TBS” means Tris-buffered saline, “TBST-X” means Tris-buffered saline containing Tween® 20 where “X” is the weight percent of Tween® 20, “SEM” means standard error of the mean, “MALDI” means matrix assisted, laser desorption ionization”, and “NMR” means nuclear magnetic resonance spectroscopy.

### General Methods:

Standard recombinant DNA and molecular cloning techniques used in the Examples are well known in the art and are described by Sambrook, J., Fritsch, E. F. and Maniatis, T., Molecular Cloning: A Laboratory Manual, Cold Spring Harbor Laboratory Press, Cold Spring Harbor, N.Y., 1989, by T. J. Silhavy, M. L. Bennan, and L. W. Enquist, Experiments with Gene Fusions, Cold Spring Harbor Laboratory, Cold Spring Harbor, N.Y., 1984, and by Ausubel, F. M. et al., Current Protocols in Molecular Biology, Greene Publishing Assoc. and Wiley-Interscience, N.Y., 1987.

Materials and methods suitable for the maintenance and growth of bacterial cultures are also well known in the art. Techniques suitable for use in the following Examples may be found in Manual of Methods for General Bacteriology, Phillipp Gerhardt, R. G. E. Murray, Ralph N. Costilow, Eugene W. Nester, Willis A. Wood, Noel R. Krieg and G. Briggs Phillips, eds., American Society for Microbiology, Washington, D.C., 1994, or by Thomas D. Brock in Biotechnology: A Textbook of Industrial Microbiology, Second Edition, Sinauer Associates, Inc., Sunderland, Mass., 1989. All reagents and materials used for the growth and maintenance of bacterial cells were obtained from Aldrich Chemicals (Milwaukee, Wis.), BD Diagnostic Systems (Sparks, Md.), Life Technologies (Rockville, Md.), or Sigma Chemical Company (St. Louis, Mo.), unless otherwise specified.

### Phage Display Peptide Libraries:

Three phage display peptide libraries were used in the following Examples. The Ph.D.-12™ Phage Display Peptide Library was purchased from New England Biolabs (Beverly, Mass.). This kit is based on a combinatorial library of random peptide 12-mers fused to a minor coat protein (pIII) of M13 phage. The displayed peptide is expressed at the N-terminus of pIII, such that after the signal peptide is cleaved, the first residue of the coat protein is the first residue of the displayed peptide. The Ph.D.-12™ library consists of approximately 2.7×10⁹ sequences.

Two phage display peptide libraries, one containing 15-mer random peptide sequences and the other containing 20-mer random peptide sequences, were prepared using the method described by Kay et al. (Combinatorial Chemistry & High Throughput Screening, Vol. 8:545-551 (2005)). This method is a modification of the method reported by Sidhu et al. (Methods in Enzymology 328:333-363 (2000)) in which E. coli strain CJ236 (dut⁻ ung⁻) is used to generate uridine-containing single-stranded phagemid DNA (U-ssDNA). This DNA is used as a template for second-strand synthesis using an oligonucleotide, not only as a primer of the second strand, but also to insert encoding random amino acids. Upon completion of second strand synthesis, the double stranded DNA is transformed into a wild-type strain. Any U-ssDNA is degraded by the host cell, thus leaving only the recombinant strand to generate phage particles. This method can be utilized to generate peptide fusions or mutations to the M13 coat proteins. The method of Kay et al. uses an amber stop codon at beginning of gene III. Oligonucleotides containing randomized stretches of DNA sequence are annealed to the single-stranded phage genome, such that the randomized region aligns with the stop codon. The ssDNA is enzymatically converted to covalently-closed, circular dsDNA and subsequently electroporated into a non-suppressor strain of E. coli. The newly synthesized DNA strand (minus strand) serves as the template for generation of the plus strand in the host cell, which is utilized for transcription/translation of viral genes and is packaged into the virus particle.

The titers for the resulting 15-mer and 20-mer libraries were 4.1×10¹² pfu/mL and 4.2×10¹² pfu/mL, respectively.

A sample containing approximately 4×10¹⁰ pfu of the phage from the library of interest was used in each experiment. The sample of the phage library was first pretreated to remove skin and plastic-binding clones. To remove skin-binding clones, the sample of the phage library was incubated for 1 h at room temperature with a sample of pig skin in a unique pig skin-bottom 96-well apparatus, which was created by applying one layer of Parafilm under the top 96-well block of a Minifold I Dot-Blot System (Schleicher & Schuell, Inc., Keene, N.H.), adding a layer of hairless pig skin on top of the Parafilm® cover, and then tightening the apparatus. The pig skin was purchased from a local supermarket and stored at −80° C. Before use, the skin was placed in deionized water to thaw, and then blotted dry using a paper towel. The surface of the skin was wiped with 90% isopropanol, and then rinsed with deionized water. After exposure to the pig skin, the phage sample was transferred to a polystyrene, 6-well cell culture cluster (Corning Inc., Acton, Mass.; Cat. No. 3526) and incubated for 1 h at room temperature to remove plastic-binding clones.

### Examples 1-3

### Identification of Hair Conditioner-Resistant Hair-Binding Peptides

The purpose of these Examples was to demonstrate the method of identifying hair conditioner-resistant hair-binding peptides, from three random phage display peptide libraries.

The hair samples used were 6-inch (15 cm) long pieces of medium brown human hairs, obtained from International Hair Importers and Products (Bellerose, N.Y.). The hairs were placed in 90% isopropanol for 30 min at room temperature and then washed 5 times for 10 min each with deionized water. The hairs were air-dried overnight at room temperature. The hairs were cut to a length of 1 cm and 10-20 hairs were placed into a microcentrifuge tube.

The phage sample, pretreated as described above to remove skin and plastic-binding clones, was added to the tube containing the hair sample and the mixture was incubated at room temperature for 1 h. The phage solution was removed and the hair sample was incubated in undiluted hair conditioner (Dove® Extra Volume Conditioner; Unilever, obtained from a local supermarket) for 5 min at room temperature. The hairs were then washed six times with TBST-0.5% buffer. After the washes, the hairs were transferred to a new tube, elution buffer, consisting of 1 mg/mL BSA in 0.2 M glycine-HCl, pH 2.2, was added, and the hair was incubated for 10 min. Then, neutralization buffer consisting of 1 M Tris-HCl, pH 9.2, was added to the tube. The phages that were eluted and those still bound to the hairs were amplified by adding fresh host cells (E. coli ER2338). The amplified and isolated phage was contacted with a fresh hair sample and the biopanning procedure was repeated two more times for each library.

After the third biopanning round, random single phage clones were selected and single plaque lysates were prepared following the manufacture's instructions (New England Biolabs) and the single stranded phage genomic DNA was purified using the QIAprep Spin M13 Kit (Qiagen; Valencia, Calif.) and sequenced at the DuPont Sequencing Facility using −96 gill sequencing primer (5′-CCCTCATAGTTAGCGTAACG-3′), given as SEQ ID NO:7. The displayed peptide is located immediately after the signal peptide of gene III. The amino acid sequences of the hair conditioner-resistant, hair-binding phage-peptides, identified from the three phage libraries after three biopanning rounds, are given in Table 1.

TABLE 1

Amino Acid Sequences of Hair-Conditioner
Resistant Hair-Binding Phage-Peptides

| | | | | SEQ | |
|---|---|---|---|---|---|
| | Phage | Clone | Amino Acid | ID | |
| Example | Library | ID | Sequence | NO: | Frequency¹ |
| 1 | 20-mer | HCP.1 | THSTHNHGSPRH | 1 | 39 |
| | | | TNADAGNP | | |
| 1 | 20-mer | HCP.2 | QQHKVHHQNPDR | 2 | 15 |
| | | | STQDAHHS | | |
| 2 | 15-mer | HCP.5 | HHGTHHNATKQK | 3 | 36 |
| | | | NHV | | |
| 2 | 15-mer | HCP.6 | STLHKYKSQDPTP | 4 | 17 |
| | | | HH | | |
| 3 | 12-mer | HCP.9 | SVSVGMKPSPRP | 5 | 16 |

¹The frequency represents the number of identical sequences that occurred out of 95 random sequenced clones.

### Example 4

### Specificity of Hair Conditioner-Resistant Hair-Binding Peptides

The purpose of this Example was to demonstrate the specificity of the hair conditioner-resistant hair-binding peptides that were identified in Examples 1-3 using an ELISA procedure.

The hair-binding peptides HCP.1 (SEQ ID NO:1) and HCP.6 (SEQ ID NO:4) were used in this Example along with a control peptide, an unrelated skin-binding peptide, Skin 1 (Huang et al., U.S. Patent Application Publication No. 2005/0050656), given as SEQ ID NO:8. All of the peptides were synthesized with an added lysine residue, derivatized with the fluorescent tag 5-carboxyfluorescein-aminohexyl amidite (5-FAM), at the C-terminus by SynPep (Dublin, Calif.). The sequences of the derivatized hair binding peptides HCP.1(5-FAM) and HCP.6(5-FAM) are given as SEQ ID NOs:9 and 10, respectively. The sequence of the derivatized skin-binding peptide control Skin 1(5-FAM) is given as SEQ ID NO:11.

For the assay, a unique hair or pig skin-bottom 96-well apparatus was created by applying one layer of Parafilm® under the top 96-well block of a Minifold I Dot-Blot System (Schleicher & Schuell, Inc., Keene, N.H.), adding hair or a layer of hairless pig skin on top of the Parafilm® cover, and then tightening the apparatus. The hair or skin sample in the 96-well apparatus was first blocked with SuperBlock® Blocking Buffer (Tris-buffered; Pierce Biotechnology, Rockford, Ill.) by incubating the sample for 1 h at room temperature. Then, the hair or skin sample was washed six times with wash buffer (TBST-0.5%). The fluorescein-labeled peptide, at a concentration of 20 μM in 1.0 mL of binding buffer (TBST-0.5% containing 1 mg/mL BSA), was added to each well and incubated for 30 min at 37° C. The hair or skin sample was washed six times with TBST-0.5%, and then, 1.0 mL of anti-fluorescein/Mouse IgG (Molecular Probes, Inc., Eugene, Oreg.) solution (1:1000 dilution in blocking buffer) was added per well. The samples were incubated for 1 h at room temperature and then washed six times with wash buffer. Then, 1.0 mL of Anti-Mouse IgG-HRP conjugate (Pierce Biotechnology) solution (1:1000 dilution in blocking buffer) was added to each well and the samples were incubated for 1 h at room temperature. The samples were washed six times with wash buffer, and 300 μL of TMB Substrate (Pierce Biotechnology) was added to each well. The samples were incubated for 10 min at room temperature and then a 100 μL sample from each well was taken and added to a well in a new microtiter plate. Then, 100 μL of Stop solution (2 M sulfuric acid solution) was added to each well and the absorbance of each sample was measured at a wavelength of 450 nm.

The results are presented in Table 2 as the mean ±the standard error of the mean (SEM) of duplicate independent experiments, each consisting of at least three replicates. As can been seen from the data in the table, the hair conditioner-resistant hair-binding peptides HCP.1 and HCP.6 bind to hair, but not to skin, demonstrating their binding specificity for hair. As expected, the Skin 1 peptide, used as a positive skin-binding control, had high skin-binding activity, but low hair-binding activity.

TABLE 2
Results of ELISA Determination of Specificity of Hair
Conditioner-Resistant Hair-Binding Peptides

| | | Hair | Skin |
|---|---|---|---|
| Peptide | SEQ ID NO: | A₄₅₀ ± SEM | A₄₅₀ ± SEM |
| HCP.1(5-FAM) | 9 | 0.392 ± 0.065 | 0.090 ± 0.136 |
| HCP.6(5-FAM) | 10 | 0.581 ± 0.053 | −0.009 ± 0.023 |
| Skin 1(5-FAM) | 11 | −0.001 ± 0.041 | 0.328 ± 0.146 |

### Example 5

### Binding of Hair Conditioner-Resistant Hair-Binding Peptides to Hair from a Hair Conditioner Matrix

The purpose of this Example was to demonstrate that the hair-conditioner resistant hair-binding peptides bind to hair from a hair conditioner matrix.

The same ELISA method described in Example 4 was used, except that the hair samples were assembled into bundles, instead of being cast as wells in a 96-well apparatus. To prepare the hair sample bundles, 100 pieces of 1 cm-long hairs were assembled together and taped at one end with narrow tape (3 M, St. Paul, Minn.). The hair was prepared as described in Examples 1-3 and placed into a flat-bottom block (Qiagen Science, Germantown, Md.; Cat. No. 19579).

The HCP.1(5-FAM) and HCP.6(5-FAM) hair-binding peptides, as described in Example 4, were mixed separately with undiluted hair conditioner (Dove® Extra Volume Conditioner; Unilever) using a high-shear mixer (Silverson, Model L4R7A; Silverson Machines, East Longmeadow, Mass.) for 6 min to give a final peptide concentration of 20 μM. The hair samples were blocked as described in Example 4 and then incubated in the peptide-conditioner mixtures for 30 min at 37° C. The hair samples were then washed and treated as described in Example 4. After the final wash step after the addition of the Anti-Mouse IgG-HRP conjugate, the hair bundles were transferred to new tubes and the TMB substrate was added. The hair bundles were incubated for 10 min at room temperature, and then a 100 μL sample from each tube was taken and added to a well in a microtiter plate. Then, 100 μL of Stop solution (2 M sulfuric acid solution) was added to each well and the absorbance of each sample was measured at a wavelength of 450 nm.

The binding of the HCP.1(5-FAM) and HCP.6(5-FAM) hair-binding peptides to hair from buffer was determined using the same procedure. In addition, controls were run using the same procedure, without any hair-binding peptide present, in both hair conditioner and buffer.

The results are presented in Table 3 as the mean ±the standard error of the mean (SEM) of duplicate independent experiments, each consisting of at least three replicates. The results demonstrate that there is no significant difference in the hair-binding activities of the hair conditioner-resistant hair-binding peptides HCP.1 and HCP.6 from a hair conditioner matrix compared to buffer.

TABLE 3
Results of ELISA Determination of Binding of Hair
Conditioner-Resistant Hair-Binding Peptides to
Hair from a Hair Conditioner Matrix

| | | 100% Conditioner | Buffer |
|---|---|---|---|
| Peptide | SEQ ID NO: | A₄₅₀ ± SEM | A₄₅₀ ± SEM |
| HCP.1(5-FAM) | 8 | 1.581 ± 0.046 | 1.593 ± 0.060 |
| HCP.6(5-FAM) | 9 | 1.217 ± 0.075 | 1.420 ± 0.062 |
| Control, | — | 0.433 ± 0.054 | 0.547 ± 0.054 |
| no peptide | | | |

Experiments were also done to determine if the hair conditioner-resistant hair-binding peptides were also shampoo resistant. For these experiments, the hair, after contacting with the hair-binding peptide (HCP.1 or HCP.6), was washed with a solution containing 30% shampoo (Pantene Pro-V, Sheer Volume, Proctor & Gamble, Cincinnati, Ohio) and the binding activity was determined using the ELISA method described above and compared to that obtained with buffer washes. The shampoo wash resulted in almost total loss of binding activity, indicating that the hair conditioner-resistant hair-binding peptides are not shampoo resistant.

### Example 6

### Stability of Hair Conditioner-Resistant Hair-Binding Peptides in a Hair Conditioner Matrix

The purpose of this Example was to demonstrate the stability of the hair conditioner-resistant hair-binding peptides in a hair conditioner matrix.

Separate mixtures of the hair-binding peptides HCP.1 and HCP.6 in hair conditioner were prepared as described in Example 5. For purposes of comparison, solutions of the hair-binding peptides in buffer were used. All the solutions were stored at room temperature and the binding activity of the peptides was determined using the ELISA procedure described in Example 5 using samples taken at different periods of time. Controls were also run with buffer and hair conditioner that did not contain the hair-binding peptide.

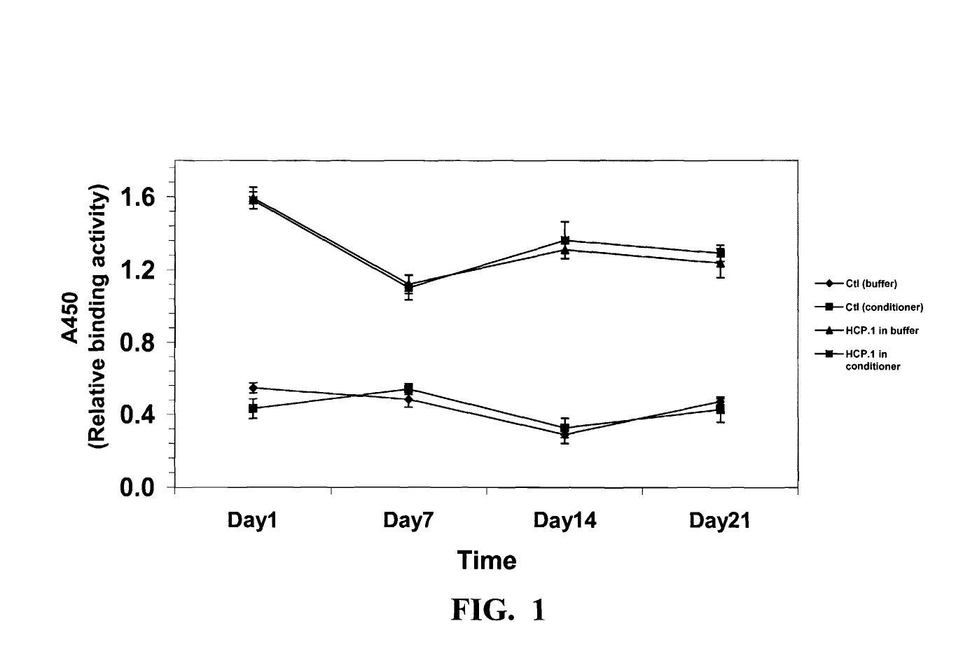

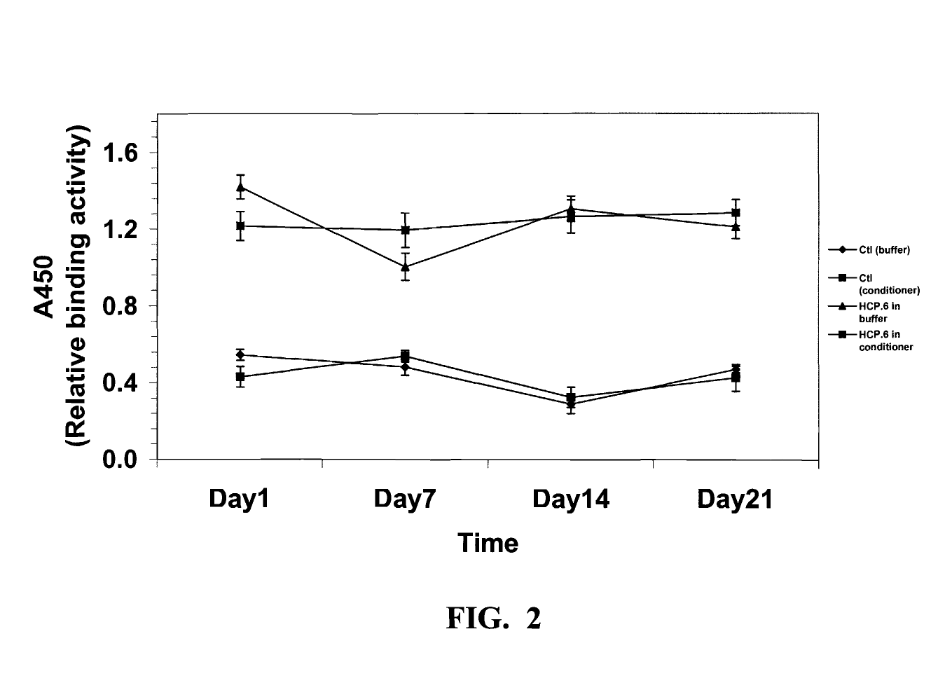

The results obtained for peptides HCP.1 and HCP.6 are shown in FIGS. 1 and 2, respectively. The results in the figures show that there was no significant decrease in the hair-binding activity of the two hair conditioner-resistant hair-binding peptides after 21 days in the hair conditioner matrix.

### Example 7 (Prophetic)

### Preparation of a Peptide-Based Hair Conditioner

The purpose of this prophetic Example is to describe how to prepare a peptide-based hair conditioner by coupling the hair conditioner-resistant hair-binding, cysteine-attached HCP.1 peptide, given as SEQ ID NO:12, with octylamine using the heterobifunctional cross-linking agent 3-maleimidopropionic acid N-hydroxysuccinimide ester.

Octylamine (from Aldrich) is diluted by adding 11.6 mg to 0.3 mL of DMF. This diluted solution is added to a stirred solution containing 25 mg of 3-maleimidopropionic acid N-hydroxysuccinimide ester (Aldrich) and 5 mg of diisopropylethylamine (Aldrich) in 0.2 mL of DMF in a 5 mL round bottom flask. The reaction mixture will turn turbid immediately and then become clear several minutes later. The solution is stirred for another 4 h. Then, the solution is dried under high vacuum. The product, octylamine-coupled maleimidopropionate, is purified by column chromatography using a Silica gel 60 (EMD Chemicals, formerly EM Science, Gibbstown, N.J.) column and DMF/ether as the eluent.

Approximately 12 mg of the above product is placed into a 5 mL round bottom flask and 85 mg of cysteine-attached HCP.1 peptide (SynPep, Dublin, Calif.), given as SEQ ID NO:12, and 0.5 mL of 0.1 M phosphate buffer at pH 7.2 are added. The cysteine-attached HCP.1 peptide has a cysteine attached to the C-terminal end of the peptide sequence of the hair-binding HCP.1 peptide (SEQ ID NO: 1). The mixture is stirred at room temperature for 6 h. The final product is purified by extraction with water/ether. The product is analyzed using liquid chromatography-mass spectrometry (LC-MS).

### Example 8 (Prophetic)

### Preparation of a Peptide-Based Hair Conditioner

The purpose of this prophetic Example is to prepare a peptide-based hair conditioner by coupling the hair conditioner-hair-binding peptide HCP.1 to an octadecyl alkyl chain.

Octadecylisocyanate (70 mg, Aldrich, CAS No. 112-96-9) is dissolved in 5 mL of N,N′-dimethylformamide (DMF) and is added to a solution of unprotected HCP.1 peptide having an added cysteine residue on the C-terminal end (from SynPep), given as SEQ ID NO:12, which is dissolved (150 mg) in 10 mL of DMF. Triethylamine (30 mg) is added to catalyze the reaction. The solution is stirred at room temperature for 120 h. The solvent is evaporated to yield an off-white, crystalline powder product. The product is analyzed by liquid chromatography and MALDI mass spectrometry.

### Example 9 (Prophetic)

### Preparation of a Peptide-Based Hair Colorant

The purpose of this prophetic Example is to prepare a peptide-based hair colorant by covalently attaching the hair conditioner-resistant hair-binding peptide HCP.1 (SEQ ID NO:1) to Disperse Orange 3 dye. The dye is first functionalized with isocyanate and then is reacted with the HCP.1 peptide.

### Functionalization of Disperse Orange 3:

In a dry box, 14.25 g of Disperse Orange 3 (Aldrich) is suspended in 400 mL of dry THF in an addition funnel. A 2-liter, four-neck reaction flask (Corning Inc., Corning, N.Y.; part no. 1533-12), containing a magnetic stir bar, is charged with 200 mL of dry toluene. The flask is fitted with a cold finger condenser (Corning Inc., part no. 1209-04) and with a second cold finger condenser with an addition funnel, and is placed on an oil bath in a hood.

Phosgene (25.4 mL) is condensed into the reaction flask at room temperature. After phosgene addition is complete, the temperature of the oil bath is raised to 80° C. and the Disperse Orange 3 suspension is added to the reaction flask dropwise in 100 mL increments over 2 h, while monitoring the reaction temperature and gas discharge from the scrubber. The temperature is maintained at or below 64° C. throughout the addition. After addition is complete, the reactants are heated at 64° C. for 1 h and then allowed to cool to room temperature with stirring overnight.

The reaction solvents are vacuum-distilled to dryness, while maintaining the contents at or below 40° C., and vacuum is maintained for an additional hour. The reaction flask is transferred to a dry box; the product is collected and dried overnight. The desired product is confirmed by proton NMR.

Coupling of Isocyanate Functionalized Dye with HCP.1 Hair-Binding Peptide:

Isocyanate functionalized Disperse Orange 3 [(2-(4-isocyantophenyl)-1-(4-nitrophenyl)diazene](16 mg), prepared as described above, is dissolved in 5 mL of DMF and added to a solution containing 75 mg of non-protected HCP.1 peptide (SEQ ID NO:1), from SynPep, dissolved in 10 mL of DMF. Triethylamine (30 mg) is added to catalyze the reaction. The solution is stirred at room temperature for 24 h. The solvent is evaporated yielding a dark red-brown powder product. The product is analyzed by MALDI mass spectrometry to confirm adduct formation.

### Example 10 (Prophetic)

### Coloring Hair Using a Peptide-Based Hair Colorant in a Hair Conditioner Matrix

The purpose of this prophetic Example is to describe how to test the coloring of a sample of natural white hair using a peptide-based hair colorant in a hair conditioner matrix.

A hair coloring composition is prepared by mixing the peptide-based hair colorant prepared as described in Example 9 with a hair conditioner. The peptide-based hair colorant (100 mg) is mixed with 10 mL of Dove® Extra Volume Conditioner using a high sheer mixer. A bundle of natural white hair (approximately 100-1000 hairs) (from International Hair Importers and Products Inc.) is immersed in the peptide-based hair colorant-conditioner mixture for 10 min with stirring. The hair is then cleaned by mixing with 10 mL of 50% Pantene Pro V shampoo for 5 min and then rinsed with distilled water to remove the shampoo. The hair is dried at room temperature and rinsed at least 5 times with distilled water. The color of the hair will be orange.

1. A method for identifying a hair conditioner-resistant hair-binding peptide comprising:
a) providing a combinatorial library of DNA associated peptides;
b) contacting the library of (a) with a hair sample wherein the hair complexes with the DNA associated peptides to form a reaction solution comprising DNA associated peptide-hair complexes;
c) isolating the DNA associated peptide-hair complexes of (b) from the reaction solution;
d) contacting the isolated DNA associated peptide-hair complexes of (c) with a hair conditioner matrix to form a conditioning solution wherein the concentration of the hair conditioner matrix is at least about 10% of full strength concentration;
e) isolating the DNA associated peptide-hair complexes of (d) from the conditioning solution;
f) amplifying the DNA encoding the peptide portion of the DNA associated peptide-hair complexes of (e); and
g) sequencing the amplified DNA of (f) encoding a conditioner resistant hair-binding peptide wherein the conditioner-resistant hair-binding peptide is identified.

2. A method according to claim 1 wherein after step (e):
i) peptides of the DNA associated peptide-hair complexes are contacted with an eluting agent whereby a portion of DNA associated peptides are eluted from the hair and a portion of DNA associated peptides remain complexed; and
ii) the eluted or complexed DNA associated peptides of (ii) are subjected to steps (f) and (g).

3. A method according to either of claims 1 or 2 wherein the DNA encoding the peptides is amplified by a process selected from the group consisting of:
a) amplifying DNA comprising a peptide coding region by polymerase chain reaction; and
b) infecting a host cell with a phage comprising DNA encoding the peptide and growing said host cell in an appropriate growth medium.

4. A method according to either of claims 1 or 2 wherein the peptides encoded by the amplified DNA of step (f) are contacted with a fresh hair sample and steps (b) through (f) are repeated one or more times.

5. A method according to claim 1 wherein step (d) is repeated one or more times.

6. A method according to claim 1 wherein the combinatorial library of DNA associated peptides is provided in a hair conditioner matrix and is contacted with a hair sample to form a reaction solution comprising DNA associated peptide-hair complexes, wherein the concentration of the hair conditioner matrix is at least about 10% of full strength concentration.

7. A method according to claim 1 wherein the combinatorial library of DNA associated peptides is provided in a hair conditioner matrix and is contacted with a hair sample to form a reaction solution comprising DNA associated peptide-hair complexes, wherein the concentration of the hair conditioner matrix is at least about 10% of full strength concentration and wherein steps (d) and (e) are optionally deleted.

8. A method according to claim 1 wherein the combinatorial library of DNA associated peptides is generated by a method selected from the group consisting of phage display, bacterial display, and yeast display.

9. A method according to claim 1 wherein the combinatorial library of DNA associated peptides is optionally contacted with a non-target either prior to or simultaneously with contacting the hair sample to remove peptides that bind to the non-target.

10. A method according to claim 1 wherein the concentration of the hair conditioner matrix is at least about 20% of full strength concentration.

11. A method according to claim 1 wherein the concentration of the hair conditioner matrix is at least about 50% of full strength concentration.

12. A method according to claim 1 wherein the concentration of the hair conditioner matrix is at least about 75% of full strength concentration.

13. A method according to claim 1 wherein the hair conditioner matrix is undiluted.

14. A method according to claim 2 wherein the eluting agent is selected from the group consisting of acid, base, salt solution, water, ethylene glycol, dioxane, thiocyanate, guanidine, and urea.

15. A hair conditioner-resistant hair-binding peptide identified by a process comprising the steps of:
a) providing a combinatorial library of DNA associated peptides;
b) contacting the library of (a) with a hair sample wherein the hair complexes with the DNA associated peptides to form a reaction solution comprising DNA associated peptide-hair complexes;
c) isolating the DNA associated peptide-hair complexes of (b) from the reaction solution;
d) contacting the isolated DNA associated peptide-hair complexes of (c) with a hair conditioner matrix to form a conditioning solution wherein the concentration of the hair conditioner matrix is at least about 10% of full strength concentration;
e) isolating the DNA associated peptide-hair complexes of (d) from the conditioning solution;
f) amplifying the DNA encoding the peptide portion of the DNA associated peptide-hair complexes of (e); and
g) sequencing the amplified DNA of (f) encoding a conditioner resistant hair-binding peptide wherein the conditioner-resistant hair-binding peptide is identified.

16. A hair conditioner-resistant hair-binding peptide selected from the group consisting of SEQ ID NO:1, SEQ ID NO:2, SEQ ID NO:3, SEQ ID NO:4, SEQ ID NO:5, and SEQ ID NO:12.

17. A diblock, peptide-based hair benefit agent having the general structure (HCPₘ)ₙ—BA, wherein;
a) HCP is a hair conditioner-resistant hair-binding peptide;
b) BA is a benefit agent;
c) m ranges from 1 to about 100; and
d) n ranges from 1 to about 50,000.

18. A triblock, peptide-based hair benefit agent having the general structure [(HCP_{X}—S)ₘ]ₙ—BA, wherein;
a) HCP is a hair conditioner-resistant hair-binding peptide;
b) BA is a benefit agent;
c) S is a spacer;
d) x ranges from 1 to about 10;
e) m ranges from 1 to about 100; and
f) n ranges from 1 to about 50,000.

19. A diblock, peptide-based benefit agent according to claim 17 wherein the benefit agent is a hair conditioning agent.

20. A triblock, peptide-based benefit agent according to claim 18 wherein the benefit agent is a hair conditioning agent.

21. A diblock, peptide-based benefit agent according to claim 17 wherein the benefit agent is a coloring agent.

22. A triblock, peptide-based benefit agent according to claim 18 wherein the benefit agent is a coloring agent.

23. A peptide-based benefit agent according to any of claims 17-22 wherein the hair conditioner-resistant hair-binding peptide is isolated by a process comprising the steps of:
a) providing a combinatorial library of DNA associated peptides;
b) contacting the library of (a) with a hair sample wherein the hair complexes with the DNA associated peptides to form a reaction solution comprising DNA associated peptide-hair complexes;
c) isolating the DNA associated peptide-hair complexes of (b) from the reaction solution;
d) contacting the isolated DNA associated peptide-hair complexes of (c) with a hair conditioner matrix to form a conditioning solution wherein the concentration of the hair conditioner matrix is at least about 10% of full strength concentration;
e) isolating the DNA associated peptide-hair complexes of (d) from the conditioning solution;
f) amplifying the DNA encoding the peptide portion of the DNA associated peptide-hair complexes of (e); and
g) sequencing the amplified DNA of (f) encoding a hair conditioner resistant hair-binding peptide wherein the hair conditioner-resistant hair-binding peptide is identified.

24. A peptide-based benefit agent according to any of claims 17-22 wherein the hair conditioner-resistant hair-binding peptide is from about 7 amino acids to about 25 amino acids in length.

25. A peptide-based benefit agent according to any of claims 17-22 wherein the hair conditioner-resistant hair-binding peptide is from about 12 amino acids to about 20 amino acids in length.

26. A peptide-based benefit agent according to any of claims 17-22 wherein the hair conditioner-resistant hair-binding peptide further comprises at least one cysteine residue on at least one end of the peptide selected from the group consisting of:
a) the N-terminal end; and
b) the C-terminal end.

27. A peptide-based benefit agent according to any of claims 17-22 wherein the hair conditioner-resistant hair-binding peptide further comprises at least one lysine residue on at least one end of the peptide selected from the group consisting of:
a) the N-terminal end; and
b) the C-terminal end.

28. (canceled)

29. A peptide-based benefit agent according to claim 19 or 20 wherein the hair conditioning agent is selected from the group consisting of octylamine, stearyl amine, behenyl alcohol, vinyl group terminated siloxanes, vinyl group terminated silicone, vinyl group terminated methyl vinyl siloxanes, vinyl group terminated methyl vinyl silicone, hydroxyl terminated siloxanes, hydroxyl terminated silicone, amino-modified silicone derivatives, [(aminoethyl)amino]propyl hydroxyl dimethyl siloxanes, [(aminoethyl)amino]propyl hydroxyl dimethyl silicones, alpha-tridecyl-omega-hydroxy-poly(oxy-1,2-ethanediyl), amodimethicone, and nanoparticles.

30. A peptide-based benefit agent according to claim 21 or 22 wherein the coloring agent is selected from the group consisting of D&C Yellow 1, D&C Yellow 3, HC Yellow 6, HC Yellow 8, D&C Blue 1, HC Blue 1, HC Brown 2, HC Red 5, 2-nitro-paraphenylenediamine, N,N-hydroxyethyl-2-nitro-phenylenediamine, 4-nitro-indole, iron oxides, titanium dioxide, carbon black, carbon nanotubes, metal nanoparticles, semiconductor nanoparticles, and colored microspheres.

31. A peptide-based benefit agent according to claim 30 wherein the colored microspheres are comprised of materials selected from the group consisting of polystyrene, polymethylmethacrylate, polyvinyltoluene, styrene/butadiene copolymer, and latex; and wherein the microspheres have a diameter of about 10 nanometers to about 2 microns.

32. A peptide-based benefit agent according to any of claims 18, 20, or 22 wherein the spacer is selected from the group consisting of ethanol amine, ethylene glycol, polyethylene with a chain length of 6 carbon atoms, polyethylene glycol with 3 to 6 repeating units, phenoxyethanol, propanolamide, butylene glycol, butyleneglycolamide, propyl phenyl, ethyl alkyl chain, propyl alkyl chain, hexyl alkyl chain, steryl alkyl chains, cetyl alkyl chains, and palmitoyl alkyl chains.

33. A peptide-based benefit agent according to any of claims 18, 20, or 22 wherein the spacer is a peptide comprising amino acids selected from the group consisting of proline, lysine, glycine, alanine, serine, and mixtures thereof.

34. A peptide-based benefit agent according to any of claims 18, 20, or 22 wherein the spacer is a peptide comprising an amino acid sequence selected from the group consisting of SEQ ID NO:6, SEQ ID NO:13, SEQ ID NO:14, and SEQ ID NO:15.

35. A hair care product composition comprising an effective amount of the peptide-based benefit agent of claim 17 or 18.

36. A hair coloring product composition comprising an effective amount of the peptide-based benefit agent of claim 21 or 22.

37. A cosmetic product composition comprising an effective amount of the peptide-based benefit agent of claim 21 or 22.

38. A hair coloring product composition comprising an effective amount of the peptide-based benefit agent of claim 19 or 20.

39. A hair conditioning product composition comprising an effective amount of the peptide-based benefit agent of claim 19 or 20.

40. A method for forming a protective layer of a peptide-based conditioner on hair comprising applying the composition of claim 39 to the hair and allowing the formation of said protective layer.

41. A method for coloring hair comprising applying the composition of claim 38 to the hair for a period of time sufficient to cause coloration of the hair.

42. A method for coloring eyebrows or eyelashes comprising applying the composition of claim 37 to eyebrow or eyelashes.

43. A method for coloring hair, eyebrows or eyelashes comprising the steps of:
a) providing a hair coloring composition comprising a hair colorant selected from the group consisting of:
i) (HCPₘ)ₙ—C; and
ii) [(HCP_{X}—S)ₘ]ₙ—C
wherein:
1) HCP is a hair conditioner-resistant hair-binding peptide;
2) C is a coloring agent;
3) n ranges from 1 to about 50,000;
4) S is a spacer;
5) m ranges from 1 to about 100; and
6) x ranges from 1 to about 10;
and wherein the hair conditioner-resistant hair-binding peptide is selected by a method comprising the steps of:
A) providing a combinatorial library of DNA associated peptides;
B) contacting the library of (A) with a hair sample wherein the hair complexes with the DNA associated peptides to form a reaction solution comprising DNA associated peptide-hair complexes;
C) isolating the DNA associated peptide-hair complexes of (B) from the reaction solution;
D) contacting the isolated DNA associated peptide-hair complexes of (C) with a hair conditioner matrix to form a conditioning solution wherein the concentration of the hair conditioner matrix is at least about 10% of full strength concentration;
E) isolating the DNA associated peptide-hair complexes of (D) from the conditioning solution;
F) amplifying the DNA encoding the peptide portion of the DNA associated peptide-hair complexes of (E); and
G) sequencing the amplified DNA of (F) encoding a hair conditioner resistant hair-binding peptide wherein the hair conditioner-resistant hair-binding peptide is identified; and
b) applying the hair coloring composition of (a) to hair, eyebrows or eyelashes for a time sufficient for the hair colorant to bind to hair, eyebrows or eyelashes.

44. A method for forming a protective layer of a peptide-based conditioner on hair comprising the steps of:
a) providing a hair care composition comprising a hair conditioner selected from the group consisting of:
i) (HCPₘ)ₙ—HCA; and
ii) [(HCP_{X}—S)ₘ]ₙ—HCA
wherein:
1) HCP is a hair conditioner-resistant hair-binding peptide;
2) HCA is a hair conditioning agent;
3) n ranges from 1 to about 50,000;
4) S is a spacer;
5) m ranges from 1 to about 100; and
6) x ranges from 1 to about 10;
and wherein the hair conditioner-resistant hair-binding peptide is selected by a method comprising the steps of:
A) providing a combinatorial library of DNA associated peptides;
B) contacting the library of (A) with a hair sample wherein the hair complexes with the DNA associated peptides to form a reaction solution comprising DNA associated peptide-hair complexes;
C) isolating the DNA associated peptide-hair complexes of (B) from the reaction solution;
D) contacting the isolated DNA associated peptide-hair complexes of (C) with a hair conditioner matrix to form a conditioning solution wherein the concentration of the hair conditioner matrix is at least about 10% of full strength concentration;
E) isolating the DNA associated peptide-hair complexes of (D) from the conditioning solution;
F) amplifying the DNA encoding the peptide portion of the DNA associated peptide-hair complexes of (E); and
G) sequencing the amplified DNA of (F) encoding a conditioner resistant hair-binding peptide wherein the conditioner-resistant hair-binding peptide is identified; and
b) applying the hair care composition of (a) to hair and allowing the formation of said protective layer.


SEQUENCE LISTING (15 sequences)

SEQ ID NO: 1 - protein, 20 aa, Artificial Sequence
  Hair conditioner-resistant hair-binding peptide
THSTHNHGSPRHTNADAGNP

SEQ ID NO: 2 - protein, 20 aa, Artificial Sequence
  Hair conditioner-resistant hair-binding peptide
QQHKVHHQNPDRSTQDAHHS

SEQ ID NO: 3 - protein, 15 aa, Artificial Sequence
  Hair conditioner-resistant hair-binding peptide
HHGTHHNATKQKNHV

SEQ ID NO: 4 - protein, 15 aa, Artificial Sequence
  Hair conditioner-resistant hair-binding peptide
STLHKYKSQDPTPHH

SEQ ID NO: 5 - protein, 12 aa, Artificial Sequence
  Hair conditioner-resistant hair-binding peptide
SVSVGMKPSPRP

SEQ ID NO: 6 - protein, 8 aa, Artificial Sequence
  Caspase 3 cleavage site
LESGDEVD

SEQ ID NO: 7 - DNA, 20 bp, Artificial Sequence
  Primer
ccctcatagttagcgtaacg

SEQ ID NO: 8 - protein, 12 aa, Artificial Sequence
  Skin-binding peptide
TPFHSPENAPGS

SEQ ID NO: 9 - protein, 21 aa, Artificial Sequence
  Fluorescein-labeled, hair conditioner-resistant hair-binding peptide
THSTHNHGSPRHTNADAGNPK

SEQ ID NO: 10 - protein, 16 aa, Artificial Sequence
  Fluorescein-labeled, hair conditioner-resistant hair-binding peptide
STLHKYKSQDPTPHHK

SEQ ID NO: 11 - protein, 13 aa, Artificial Sequence
  Fluorescein-labeled, skin-binding peptide
TPFHSPENAPGSK

SEQ ID NO: 12 - protein, 21 aa, Artificial Sequence
  Cysteine-attached, hair conditioner-resistant hair-binding peptide
THSTHNHGSPRHTNADAGNPC

SEQ ID NO: 13 - protein, 37 aa, Artificial Sequence
  Peptide spacer
TSTSKASTTTTSSKTTTTSSKTTTTTSKTSTTSSSST

SEQ ID NO: 14 - protein, 22 aa, Artificial Sequence
  Peptide spacer
GQGGYGGLGSQGAGRGGLGGQG

SEQ ID NO: 15 - protein, 10 aa, Artificial Sequence
  Peptide spacer
GPGGYGPGQQ